(19) United States
(12) Patent Application Publication
(10) Pub. No.: US 2020/0171159 A1
(43) Pub. Date: Jun. 4, 2020

(54) SELECTIVE DESTRUCTION OF CELLS

(71) Applicant: YISSUM RESEARCH DEVELOPMENT COMPANY OF THE HEBREW UNIVERSITY OF JERUSALEM LTD., Jerusalem (IL)

(72) Inventors: Abraham LOYTER, Jerusalem (IL); Assaf FRIEDLER, Mevaseret Zion (IL)

(21) Appl. No.: 16/614,981

(22) PCT Filed: Oct. 25, 2017
(86) PCT No.: PCT/IL2017/051169

§ 371 Date: Nov. 19, 2019

Related U.S. Application Data
(60) Provisional application No. 62/509,119, filed on May 21, 2017.

(51) Int. Cl. A61K 47/64; A61K 38/46; A61K 31/713; A61K 47/54; A61K 47/68; A61K 38/45; A61K 39/395; A61K 9/51; A61K 38/10; A61P 35/00; A61K 35/76

(52) U.S. Cl. CPC A61K 47/64; A61K 38/465; A61K 31/713; A61K 47/549; A61K 47/6883; A61K 35/76; A61K 39/3955; A61K 9/5169; A61K 9/5123; A61K 38/10; A61P 35/00; A61K 38/45

(57) ABSTRACT

The present invention provides compositions and methods for inducing DNA breaks in specifically-targeted cells, in particular cancer and HIV-infected cells, thereby promoting cell death.

### FIELD OF THE INVENTION

The present invention relates to methods and composition for inducing DNA breaks in cells, thereby promoting cell death. Specifically, the present invention relates to the use of integrase enzyme complexes, alone or together with an integration-promoting agent, for inducing double-stranded DNA breaks in nuclear DNA in specific cell populations.

### BACKGROUND OF THE INVENTION

Retroviruses are a large and diverse family of RNA viruses that make a DNA copy of their RNA genome after infection of a host cell. The human immunodeficiency virus (HIV) is a lentivirus (a subgroup of retrovirus) that causes the acquired immunodeficiency syndrome (AIDS), a condition in humans in which progressive failure of the immune system allows life-threatening opportunistic infections and cancers to thrive. Without treatment, average survival time after infection with HIV is estimated to be 9 to 11 years, depending on the HIV subtype. Infection with HIV occurs by the transfer of blood, semen, vaginal fluid, pre-ejaculate, or breast milk Within these bodily fluids, HIV is present as both free virus particles and virus within infected immune cells.

HIV infects vital cells in the human immune system such as helper T cells (specifically CD4+ T cells), macrophages, and dendritic cells. HIV infection leads to low levels of CD4+ T cells through a number of mechanisms, including apoptosis of uninfected bystander cells, direct viral killing of infected cells, and killing of infected CD4+ T cells by CD8 cytotoxic lymphocytes that recognize infected cells. When CD4+ T cell numbers decline below a critical level, cell-mediated immunity is lost, and the body becomes progressively more susceptible to opportunistic infections.

An essential step in the replication cycle of HIV-1 and other retroviruses is the integration of the reversed-transcribed viral DNA into the host DNA. The RNA genome of progeny virions and the template for translation of viral proteins are made when the integrated viral DNA is transcribed. HIV integration is the insertion of HIV genetic material into the genome of the infected cell. The process of HIV integration involves six sequential steps: binding of HIV Integrase (IN) to HIV DNA, processing of the HIV DNA 3′ ends, translocation of HIV integrase to the host cell nucleus (nuclear translocation), binding of the pre-integration complex to the host DNA, transfer of HIV DNA into the host DNA (Strand Transfer), and repair of the gaps formed in the strand transfer process (“Gap Repair”).

Li et al. (“Retroviral DNA integration: reaction pathway and critical intermediates”, The EMBO Journal, 2006, Vol. 25, pages 1295-1304) shows that HIV-1 integrase forms stable synaptic complexes in which a tetramer of integrase is stably associated with a pair of viral DNA ends. The viral DNA is processed within these complexes, which go on to capture the target DNA and integrate the viral DNA ends.

Hayouka et al. (“Peptides Derived From HIV-1 Rev Inhibit HIV-1 Integrase in a Shiftide Mechanism”, PeptideScience, 2008, Vol. 90(4), pages 481-487), describes an approach for inhibiting integrase by “shiftides”: peptides derived from its cellular binding protein LEDGF/p75, and from its viral binding protein Rev, that inhibit integrase by shifting its oligomerization equilibrium from the active dimer to an inactive tetramer.

Contreras et al. (“Suberoylanilide Hydroxamic Acid Reactivates HIV from Latently Infected Cells”, The Journal of Biological Chemistry, 2009, Vol. 284(11), pages 6782-6789) describes a method for purging the reservoir of latent proviruses, which account for the rebound in viral replication observed after highly active antiretroviral therapy treatment interruption, by suberoylanilide hydroxamic acid (SAHA).

Levin et al. (“Peptides Derived from HIV-1 Integrase that Bind Rev Stimulate Viral Genome Integration”, 2009, PLoS One, Vol. 4(1), page e4155), describes the derivation of peptides, designated INr1 and INr2, which interact with the Rev protein and blocks the Rev-integrase interaction. The authors demonstrate that INr1 and INr2 are able to abrogate the inhibitory effects exerted by Rev and Rev-derived peptides on integrase activity. Both INr1 and INr2 were found to be cell-permeable and nontoxic. Interestingly, both INr peptides stimulated virus infectivity. Furthermore, kinetics studies revealed that the INr peptides enhanced the integration process by dissociating the Rev-Integrase complex.

WO 2010/041241, entitled “HIV-1 integrase derived stimulatory peptides interfering with integrase-REV protein binding”, relates to isolated peptides comprising sequences derived from the protein integrase of HIV-1, as well as their analogs, mixtures, conjugates, and pharmaceutical compositions, targeted to selectively kill HIV-1 infected cells.

Levin et al. (“Stimulation of the HIV-1 Integrase Enzymatic Activity and cDNA Integration by a Peptide Derived from the Integrase Protein”, Biopolymers, 2010, Vol. 93, pages 740-751), describes a peptide designated INS (for IN stimulatory), which interacts with both the viral Rev and IN proteins. The authors demonstrate that this peptide is able to dissociate the inactive Rev-IN complex, and directly activates the free IN Similar to the INr1 and INr2 peptides described above, the INS peptide was found to be cell-permeable and nontoxic. The INS peptide also stimulated virus infectivity and enhanced the integration process due to its ability to dissociate the Rev-Integrase complex.

Levin et al. (“Specific eradication of HIV-1 from infected cultured cells”, AIDS Research and Therapy, 2010, Vol. 7, page 31), describes the eradication of HIV-1 infected cells by the disruption of the Rev-Integrase complex formed in these cells.

Cooray et al. (“Retrovirus and Lentivirus Vector Design and Methods of Cell Conditioning”, Methods in Enzymology, 2012, Vol. 507, pages 29-57) provides a comprehensive review on retrovirus and lentivirus vector design, production and transduction procedures into target cells.

Cooper et al. (“HIV-1 causes CD4 cell death through DNA-dependent protein kinase during viral integration”, Nature, 2013, Vol. 498, pages 376-380) suggests a model, according to which activation of DNA-dependent protein kinase (DNA-PK), after DNA double strand break and during viral integration has, a central role in CD4⁺ T-cell death (cell depletion), raising the possibility that integrase inhibitors and interventions directed towards DNA-PK may improve T-cell survival and immune function in infected individuals. This model was analyzed by Skalka (“Integration triggers death”, Nature, 2013, Vol. 498, pages 305-306), which specifically notes that it may be assumed that the integration-induced damage in chromosomal DNA, which results in cell death, is repaired, and that apoptosis seems to be a delayed response.

Roos and Kaina (“DNA damage-induced cell death: From specific DNA lesions to the DNA damage response and apoptosis”, Cancer Letters, 2013, Vol. 332, pages 237-248) review the connection between DNA lesions, DNA-damage response and apoptosis.

Gabizon et al. (“Structure—activity relationship studies using peptide arrays: the example of HIV-1 Rev-integrase interaction”, Med. Chem. Commun., 2013, Vol. 4, pages 252-259), explored the mechanism of action of INr1, INr2 and INS peptides (derived from residues IN 66-80, IN 118-128 and IN 174-188, respectively) as the basis for developing improved anti HIV-1 agents. The authors found that all three peptides abrogated the IN-Rev interaction, resulting in activation of IN and stimulation of viral DNA integration in HIV-1 infected cells, while INS also had a direct stimulatory effect on IN activity.

Margolis (“How Might We Cure HIV?”, Curr. Infect. Dis. Rep., 2014, Vol. 16, page 392) reviews an emerging concept of HIV-1/AIDS therapy, in which the state of virological latency is disrupted, followed by clearance of residual HIV and HIV-infected cells from the body.

Cancer, also known as a malignant tumor or malignant neoplasm, include a group of diseases involving abnormal cell growth with the potential to invade or spread to other parts of the body.

Viruses are by far the most abundant biological entities on Earth and they outnumber all the others put together. They infect all types of cellular life including animals, plants, bacteria and fungi. However, different types of viruses can infect only a limited range of hosts and many are species-specific. Some, such as smallpox virus for example, can infect only one species—in this case humans, and are said to have a narrow host range. Other viruses, such as rabies virus, can infect different species of mammals and are said to have a broad range. The viruses that infect plants are harmless to animals, and most viruses that infect other animals are harmless to humans. Examples of common human diseases caused by viruses include the common cold, influenza, chickenpox, and cold sores. Many serious diseases such as Ebola virus disease, AIDS, avian influenza, and SARS are caused by viruses.

Obesity is a medical condition in which excess body fat has accumulated to the extent that it may have a negative effect on health, leading to reduced life expectancy and/or increased health problems. In Western countries, people are considered obese when their body mass index (BMI), a measurement obtained by dividing a person's weight by the square of the person's height, exceeds 30 kg/m², with the range 25-30 kg/m² defined as overweight. Some East Asian countries use stricter criteria.

Specific population of cells, such as cancer cells, pathogen-infected cells and fat cells represent a wider array of cells which are often found in the human body and are causative or associated with an array of human diseases and adverse conditions. There is a need for novel remedies of high therapeutic efficacy with minimal side effects for eradication of such cells. More specifically, there is a need for targeted tools for the destruction of specific cells or cell populations in subjects of a variety of diseases or conditions.

### SUMMARY OF THE INVENTION

The present invention provides pharmaceutical composition and methods for their use in treating a variety of human diseases and conditions, in which eradication of specific cells or cell populations within a subject's body would be beneficial. More specifically, the present invention provides complexes comprising retrovirus-derived integrase enzymes, and accessory molecules, specifically targeted to their destination. Targeting is achieved for example, by complex encapsulation in cell-type-specific vehicles, specifically expression in the target destination under a selective promoter, or specifically administration to the target destination.

The present invention further provides methods and means for the specific delivery and/or specific expression of such integrase enzymes, accessory DNA, complexes and/or vehicles to predefined cells or cell populations based on the cells' unique properties, which exclude them from normal, healthy cells.

Without being bound to any theory or mechanism, it is hypothesized that by cooperation between integrase enzymes and accessory DNA and optionally accessory integration promoting agents, the integrase enzymes are translocated into the nuclei of the target cells, where they induce double-stranded breaks (DSBs) in the chromosomal DNA of the cell. It is further hypothesized that the accumulation of DSBs would exhaust the natural DNA repair capabilities of the cell, and would drive the cell to apoptosis.

The present invention therefore provides, in one aspect, a method for destroying a specific population of target cells, comprising the steps of selectively contacting the cells with a molecule of dsDNA having at least one strand capable of specific binding to the integrating enzyme, an integrating enzyme, capable of specific binding to the dsDNA molecule, entering the nucleus of a cell, and creating DSBs in the chromosomal DNA of the cell, and optionally an integration promoting agent.

According to some embodiments, the method for selectively destroying a specific population of cells, comprises the steps of: (a) targeting the cells with a complex comprising: (i) a linear molecule of double-stranded DNA (dsDNA) comprising long term repeat (LTR) sequences recognized by the integrating enzyme of (ii), and (ii) an integrating enzyme, capable of entering the nuclei of the cells after binding to the LTR sequences of the dsDNA molecule of (i) and creating multiple double strand breaks (DSBs) in the chromosomal DNA of the cells); and (b) contacting the cells with at least one integration-promoting agent.

According to some embodiments, the specific population of cells is selected from the group consisting of: cancer cells, virally-infected cells, lipocytes (adipocytes), yeast cells and bacteria cells.

In certain embodiments, the specific population of cells is a population of cancer cells or a population of HIV-infected cells. In certain embodiments, the cells are cancer cells. In certain embodiments, the cells are HIV-infected cells.

According to some embodiments, the dsDNA molecule, the integrating enzyme, or both, are selectively targeted to the specific population of cells.

According to some embodiments, selectively targeting to the specific population of cells is achieved by: i. a targeting moiety, capable of binding specifically to a molecule presented by the specific population of cells, included in said complex; ii. specific expression under a selective promoter, of the dsDNA molecule, the integrating enzyme, or both in their target destination; or iii. direct administration of the complex to the specific population of cells.

According to some embodiments, the complex further comprises a targeting moiety capable of binding specifically to a molecule presented by the specific population of cells.

According to some embodiments, direct administration to the target population of cells comprises local administration to a specific tissue or organ. Direct administration includes but is not limited to intra-tumoral injection, injection into the hepatic vain, intrathecal administration and intra-bladder injection.

In certain embodiments, the targeting moiety is an antibody or an antigen-binding fragment thereof capable of specific binding to an antigen presented by the cell. In certain embodiments, the antibody or an antigen-binding fragment thereof is an antibody directed to the human protein cluster of differentiation 24 (CD24). In certain embodiments of the method, the antibody or an antigen-binding fragment thereof is not an antibody directed to the human protein CD24. In certain embodiments, the antibody or an antigen-binding fragment thereof is an antibody directed to the human protein cluster of differentiation 20 (CD20).

In certain embodiments, the targeting moiety is connected to the complex via a linker. In certain embodiments, the linker connects the targeting moiety to the dsDNA or to the integrating enzyme. According to some embodiments, the linker comprises 1 to 30 continuous amino-acids.

In certain embodiments, the integration-promoting agent is selected from the group consisting of: INS peptide (WTAVQMAVFIHNFKRK; SEQ ID NO:1), INr2 peptide (WGSNFTSTTVKA; SEQ ID NO:3), INr1 peptide (WTHLEGKIILVAVHVA; SEQ ID NO:2), LEDGF/p75 protein (UniProt O75475), a peptide comprising SEQ ID NO:4, a peptide comprising SEQ ID NO:5, and a peptide comprising SEQ ID NO:6, and any combination thereof. Each possibility represents a separate embodiment of the invention.

In certain embodiments, the integration-promoting agent is a combination of at least two of the peptides: INS, INr1, and INr2.

In certain embodiments, the complex is further associated with at least one integration-promoting agent. According to some embodiments, the at least one integration-promoting agent is couples to the integrating enzyme. According to other embodiments, the at least one integration-promoting agent is expressed by the dsDNA. In certain embodiments, the integration-promoting agent is a mixture of INS peptide (WTAVQMAVFIHNFKRK; SEQ ID NO:1), INr2 peptide (WGSNFTSTTVKA; SEQ ID NO:3), and INr1 peptide (WTHLEGKIILVAVHVA; SEQ ID NO:2).

In certain embodiments, the dsDNA molecule encodes the integrating enzyme, the integration-promoting agent, or both. In certain embodiments, the dsDNA molecule encodes the integration-promoting agent. In certain embodiments, the dsDNA molecule encodes the integrating enzyme. In yet other embodiments, the dsDNA further encodes at least one integrating promoting agent.

Any linear dsDNA sequence comprising LTR sequences recognized by the integrating enzyme, can be used according to the present invention. dsDNA sequences according to the present invention may be naturally occurring, or non-naturally occurring molecules, and include isolated sequences, random sequences and synthetically produced sequences, or combinations thereof. Any method known in the art to isolate or synthesize dsDNA sequences may be used according to the present invention. In certain embodiments, the dsDNA molecule is a synthetic dsDNA molecule that has been prepared entirely or at least partially by chemical means.

In certain embodiments, the integrating enzyme is an integrase enzyme selected from the group consisting of HIV-1 integrase, HIV-2 integrase, an active fragment thereof, and an active analog thereof.

In certain embodiments, the method further comprises the step of contacting the cells with (i) a transfection-promoting agent, (ii) an apoptosis-promoting agent, or (iii) an antigenicity-promoting agent.

In certain embodiments, the transfection-promoting agent is capable of increasing the number of the complexes fusing with the cells, or increasing the rate of fusion between the complexes and the cells.

In certain embodiments, the antigenicity-promoting agent is selected from the group consisting of a cancer-associated antigen and a pathogen-associated antigen. In certain embodiments, the antigenicity-promoting agent reactivates a latent or dormant pathogen to express a pathogen-associated antigen.

In certain embodiments, the latent or dormant pathogen is an integrated HIV provirus, and the antigenicity-promoting agent is selected from the group consisting of an inhibitor of histone deacetylase (HDAC), suberoylanilide hydroxamic acid (SAHA), Ro5-3335 (CAS number 30195-30-3), vorinostat (CAS number 149647-78-9), panobinostat (CAS number 404950-80-7), a protein kinase C (PKC) activator, prostratin (CAS number 60857-08-1) and bryostatin (CAS number 83314-01-6).

In certain embodiments, the complex is in a lentivirus particle. In certain embodiments, the complex is not in a lentivirus particle. In certain embodiments, the complex is in a lipid-coated or a protein-coated particle.

In certain embodiments, the cells are in the body of a human subject, and targeting or contacting is administration to the subject.

In certain embodiments, the complex and the integration-promoting agent are administered systemically. In certain embodiments, the complex and the integration-promoting agent are administered simultaneously. In certain embodiments, the complex and the integration-promoting agent are administered together. In certain embodiments, the complex and the integration-promoting agent are administered separately. In certain embodiments, the complex and the integration-promoting agent are administered under different administration regimes. In certain embodiments, the complex is administered less frequently than the integration-promoting agent. In certain embodiments, the complex is administered once or twice weekly and the integration-promoting agent are administered every day or twice weekly.

The present invention thus provides, according to some embodiments, a method of selectively destroying a population of cancer cells comprising administering to a subject in need thereof, a complex comprising (i) a linear molecule of double-stranded DNA (dsDNA) comprising long term repeat (LTR) sequences recognized by the integrating enzyme of (ii), (ii) an integrating enzyme, capable of entering the nuclei of cells after binding to the LTR sequences of the dsDNA molecule of (i) and creating multiple double strand breaks (DSBs) in the chromosomal DNA of the cells, and (iii) a targeting moiety capable of binding at least one molecule presented by the cancer cells, and (b) administering to the subject at least one integration-promoting agent.

According to some embodiments, the complex is a lentivirus particle comprising the linear dsDNA molecule, an integrase, and an antibody directed against a cell surface marker.

According to some embodiments, the cell surface marker is selected from CD24 and CD20.

According to a specific embodiment, a method of selectively destroying a population of cancer cells is provided comprising administering to a subject in need thereof, a complex comprising (i) a lentivirus particle comprising a linear molecule of double-stranded DNA (dsDNA) comprising long term repeat (LTR) sequences recognized by the integrating enzyme of (ii), (ii) an integrase, capable of entering the nuclei of cells after binding to the LTR sequences of the dsDNA molecule of (i) and creating multiple double strand breaks (DSBs) in the chromosomal DNA of the cells, and (iii) an antibody capable of binding CD24 presented by the cancer cells, and (b) administering to the subject at least one integration-promoting agent selected from the group consisting of: SEQ ID Nos 1-6, and any combination thereof. Each possibility represents a separate embodiment of the invention.

The present invention further provides, in another aspect, a method for treating a disease or condition in a subject in need of such treatment, comprising the steps of administering to the subject a molecule of dsDNA having at least one strand capable of specific binding to an integrating enzyme, and an integrating enzyme, capable of specific binding to the dsDNA molecule, entering the nucleus of a cell, and creating DSBs in the chromosomal DNA of the cell.

According to some embodiments, the method for treating a disease or condition in a subject in need of such treatment, comprises the steps of (a) targeting a specific population of cells of the subject with a complex comprising (i) a linear molecule of double-stranded DNA (dsDNA) comprising long term repeat (LTR) sequences recognized by the integrating enzyme of (ii), (ii) an integrating enzyme, capable of entering the nuclei of cells after binding to the LTR sequences of the dsDNA molecule of (i) and creating multiple double strand breaks (DSBs) in the chromosomal DNA of the cells, and (b) administering to the subject at least one integration-promoting agent.

According to some embodiments, the specific population of cells is selected from the group consisting of: cancer cells, virally-infected cells, lipocytes, yeast cells and bacteria cells and the disease or condition is selected from the group consisting of cancer, viral-infection, obesity, yeast infection and bacterial infection. In certain embodiments, the disease is cancer, and the cells are cancer cells. In certain embodiments, the disease is HIV infection or AIDS and the cells are HIV-infected cells.

In certain embodiments, the dsDNA molecule, the integrating enzyme, or both, are selectively targeted to the specific population of cells.

According to some embodiments, targeting to the specific population of cells is achieved by means of: i. a targeting moiety, capable of binding specifically to a molecule presented by the specific population of cells; ii. specific expression under a selective promoter, of the dsDNA molecule, the integrating enzyme, or both in their target destination; or iii. direct administration of the complex to the specific population of cells.

According to some embodiments, the complex comprises a targeting moiety capable of binding specifically to a molecule presented by the specific population of cells.

In certain embodiments, the targeting moiety is an antibody or an antigen-binding fragment thereof capable of specific binding to an antigen presented by the cell. In certain embodiments, the antibody or an antigen-binding fragment thereof is an antibody directed to the human protein CD24. In certain embodiments of the method, the antibody or an antigen-binding fragment thereof is not an antibody directed to the human protein CD24. In certain embodiments, the antibody or an antigen-binding fragment thereof is an antibody directed to the human protein CD20.

In certain embodiments, the targeting moiety is connected to the complex via a linker. In certain embodiments, the linker connects the targeting moiety to the dsDNA or to the integrating enzyme. According to some embodiments, the linker comprises 1 to 30 continuous amino-acids.

According to some embodiments, direct administration to the target population of cells comprises local administration to a specific tissue or organ. Direct administration includes but is not limited to intra-tumoral injection, injection into the hepatic vain, intrathecal administration and intra-bladder injection.

In certain embodiments, the integration-promoting agent is selected from the group consisting of INS peptide (WTAVQMAVFIHNFKRK; SEQ ID NO:1), INr2 peptide (WGSNFTSTTVKA; SEQ ID NO:3), INr1 peptide (WTHLEGKIILVAVHVA; SEQ ID NO:2), LEDGF/p75 protein (UniProt O75475), a peptide comprising SEQ ID NO:4, a peptide comprising SEQ ID NO:5, and a peptide comprising SEQ ID NO:6, and any combination thereof. Each possibility represents a separate embodiment of the invention.

In certain embodiments, the integration-promoting agent is a combination of at least two of: INS peptide, INr1 peptide, and INr2 peptide.

In certain embodiments, the dsDNA molecule encodes the integrating enzyme, the integration-promoting agent, or both. In certain embodiments, the dsDNA molecule encodes the integration-promoting agent. In certain embodiments, the dsDNA molecule encodes the integrating enzyme. In yet other embodiments, the dsDNA further encodes at least one integrating promoting agent.

In certain embodiments, the method further comprises the step of contacting the cells with (i) a transfection-promoting agent, (ii) an apoptosis-promoting agent, or (iii) an antigenicity-promoting agent.

In certain embodiments, the transfection-promoting agent is capable of increasing the number of the complexes fusing with the cells, or increasing the rate of fusion between the complexes and the cells.

In certain embodiments, the antigenicity-promoting agent is selected from the group consisting of a cancer-associated antigen and a pathogen-associated antigen. In certain embodiments, the antigenicity-promoting agent reactivates a latent or dormant pathogen to express a pathogen-associated antigen.

In certain embodiments, the latent or dormant pathogen is an integrated HIV provirus, and the antigenicity-promoting agent is selected from the group consisting of an inhibitor of histone deacetylase (HDAC), suberoylanilide hydroxamic acid (SAHA), Ro5-3335 (CAS number 30195-30-3), vorinostat (CAS number 149647-78-9), panobinostat (CAS number 404950-80-7), a protein kinase C (PKC) activator, prostratin (CAS number 60857-08-1) and bryostatin (CAS number 83314-01-6).

In certain embodiments, the complex is in a lentivirus particle. In certain embodiments, the complex is not in a lentivirus particle. In certain embodiments, the complex is in a lipid-coated or a protein-coated particle.

In certain embodiments, the cells are in the body of a human subject, and targeting or contacting is administration to the subject.

In certain embodiments, the complex and the integration-promoting agent are administered systemically. In certain embodiments, the complex and the integration-promoting agent are administered simultaneously. In certain embodiments, the complex and the integration-promoting agent are administered together. In certain embodiments, the complex and the integration-promoting agent are administered separately. In certain embodiments, the complex and the integration-promoting agent are administered under different administration regimes. In certain embodiments, the complex is administered less frequently than the integration-promoting agent. In certain embodiments, the complex is administered once or twice weekly and the integration-promoting agent are administered every day or twice weekly.

In certain embodiments, the specific population of cells is a population of cancer cells or a population of human immunodeficiency virus (HIV)-infected cells. In certain embodiments, the cells are cancer cells. In certain embodiments, the cells are HIV-infected cells.

In certain embodiments, the disease or condition is selected from the group consisting of cancer and viral infection. In certain embodiments the disease is cancer and the cells are cancer cells. In certain embodiments, the disease or condition is viral infection and the cells are virally-infected cells. In certain embodiments, the virus in the virally-infected cells is active and/or replicating. In certain embodiments, the virus in the virally-infected cells is latent and/or dormant.

In certain embodiments, the virally-infected cell is an HIV-1 infected cell. In certain embodiments, the HIV-1 in the HIV-1 infected cell is replicating. In certain embodiments, the HIV-1 in the HIV-1 infected cell is in a dormant or latent stage or in a provirus stage. In certain embodiments, the subject is an HIV carrier. In certain embodiments, the subject has AIDS.

In certain embodiments, the cells are cancer cells and the disease or condition is cancer. In certain embodiments, the cancer cells are CD24⁺. According to some embodiments of the invention, the cancer is a metastatic cancer. According to some embodiments, the cancer is a solid cancer. According to some specific embodiments, the solid cancer is selected from the group consisting of melanoma (skin), lung, colon, breast, uterine, and renal cancer. According to specific embodiments, the cancer is selected from the group consisting of breast cancer, lung cancer, and liposarcoma. In certain embodiments, the cancer is selected from the group consisting of breast cancer, non-small-cell lung cancer, colorectal adenocarcinoma, breast adenocarcinoma cancer, and pancreatic carcinoma. Each possibility represents a separate embodiment of the invention.

According to some embodiments of the invention, the cancer is selected from the group consisting of a melanoma, a breast cancer, an ovarian cancer, a pancreatic cancer, a colorectal cancer, a colon cancer, a cervical cancer, a kidney cancer, a lung cancer, a thyroid cancer, a prostate cancer, a brain cancer, a renal cancer, a throat cancer, a laryngeal carcinoma, a bladder cancer, a hepatic cancer, a fibrosarcoma, an endometrial cells cancer, a glioblastoma, sarcoma, a myeloid, a leukemia and a lymphoma. Each possibility represents a separate embodiment of the invention.

According to other embodiments, the cancer is hematologic cancer. According to some embodiments, the hematologic cancer is myeloid leukemia, acute lymphoblastic leukemia, chronic lymphocytic leukemia, myeloproliferative diseases, multiple myeloma, or myelodysplastic syndrome. Each possibility represents a separate embodiment of the invention. According to certain embodiments, the cancer is leukemia. According to specific embodiments, the cancer is acute myeloid leukemia (AML).

The cancer amendable for treatment by the present invention includes, but is not limited to: carcinoma, lymphoma, blastoma, sarcoma, and leukemia or lymphoid malignancies. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high-grade immunoblastic NHL; high-grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. In certain embodiments, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma.

According to certain embodiments, the cancer is selected from the group consisting of adrenocortical carcinoma (ACC), chromophobe renal cell carcinoma (KICH), liver hepatocellular carcinoma (LIHC), colon and rectal adenocarcinoma (COAD, READ), pancreatic ductal adenocarcinoma (PAAD), pheochromocytoma & paraganglioma (PCPG), papillary kidney carcinoma (KIRP), lung adenocarcinoma (LUAD), head and neck squamous cell carcinoma (HNSC), prostate adenocarcinoma (PRAD), uterine corpus endometrial carcinoma (UCEC), cervical cancer (CESC), cutaneous melanoma (SKCM), mesothelioma (MESO), urothelial bladder cancer (BLCA), clear cell kidney carcinoma (KIRC), lung squamous cell carcinoma (LUSC), uterine carcinosarcoma (UCS), sarcoma (SARC), ovarian serous cystadenocarcinoma (OV), papillary thyroid carcinoma (THCA), glioblastoma multiforme (GBM), breast cancer (BRCA), lower grade glioma (LGG), and diffuse large B-cell lymphoma (DLBC). Each possibility represents a separate embodiment of the invention.

In certain embodiments, the cells are breast cancer cells. In certain embodiments, the cells are lung cancer cells. In certain embodiments, the cells are non-small-cell lung carcinoma cells.

Any linear dsDNA sequence comprising LTR sequences recognized by the integrating enzyme, can be used according to the present invention. dsDNA sequences according to the present invention may be naturally occurring, or non-naturally occurring molecules, and include isolated sequences, random sequences and synthetically produced sequences, or combinations thereof. In certain embodiments, the dsDNA molecule is a synthetic dsDNA molecule that has been prepared entirely or at least partially by chemical means. Synthetic DNA sequences may be used, for example, for modifying native DNA sequences in terms of codon usage and expression efficiency.

In certain embodiments, the dsDNA molecule is incorporated to the genome of the cells by site-specific recombination. In certain embodiments, the dsDNA molecule encodes a pro-apoptotic protein or peptide. In certain embodiments, the dsDNA molecule encodes an immunogenic protein or peptide. In certain embodiments, the protein or peptide is secreted from the cell. In certain embodiments, the protein or peptide is a protein or peptide of HIV-1. In certain embodiments, the protein or peptide of HIV-1 is gp41 or gp120. In certain embodiments, the targeting moiety specifically binds to gp41 or gp120. In certain embodiments, the targeting moiety is an antibody or an antigen-binding fragment thereof which specifically binds to gp41 or gp120.

In certain embodiments, both strands of the dsDNA molecule are capable of specific binding to the integrating enzyme. In certain embodiments, both strands of the dsDNA molecule comprise an LTR sequence recognized by the integrating enzyme. In certain embodiments, both strands of the dsDNA molecule comprise a cytosine-adenine dinucleotide near their 3′ end. In certain embodiments, both strands of the dsDNA molecule comprise a cytosine-adenine dinucleotide at a distance of 2-20 nucleotides 5′ to their 3′ end.

In certain embodiments, the dsDNA molecule is reverse-transcribed from a retrovirus-derived RNA molecule. In certain embodiments, the RNA molecule is part of a retrovirus-derived vector. In certain embodiments, the retrovirus-derived vector is specifically targeted to the cell by comprising a targeting agent, capable of specific binding to a target molecule presented by the cell. In certain embodiments, the targeting agent is an antibody capable of specific binding to an antigen presented by the cell. In certain embodiments, the retrovirus-derived vector further comprises the integrating enzyme and a reverse-transcriptase enzyme.

In certain embodiments, the dsDNA molecule is a synthetic dsDNA molecule. In certain embodiments, the synthetic dsDNA molecule is naked or encapsulated by a shell. In certain embodiments, the shell comprises a targeting agent, capable of specific binding to a target molecule presented by the cell. In certain embodiments, the targeting agent is an antibody capable of specific binding to an antigen presented by the cell. In certain embodiments, the shell further encapsulates the integrating enzyme.

In certain embodiments, the integrating enzyme is an integrase enzyme selected from the group consisting of HIV-1 integrase, HIV-2 integrase, active fragments thereof, and active analogs thereof. Each possibility represents a separate embodiment of the invention.

In certain embodiments, the integrating enzyme is part of a retrovirus-derived vector. In certain embodiments, the retrovirus-derived vector is specifically targeted to the cell by comprising a targeting agent, capable of specific binding to a target molecule presented by the cell. In certain embodiments, the targeting agent is an antibody capable of specific binding to an antigen presented by the cell. In certain embodiments, the retrovirus-derived vector further comprises the dsDNA molecule.

In certain embodiments, the integrating enzyme is encapsulated by a shell. In certain embodiments, the shell comprises a targeting agent, capable of specific binding to a target molecule presented by the cell. In certain embodiments, the targeting agent is an antibody capable of specific binding to an antigen presented by the cell. In certain embodiments, the shell further encapsulates the dsDNA molecule.

In certain embodiments, the methods described above further comprise the step of introducing to the cell at least one agent selected from the group consisting of an integration-promoting agent, an apoptosis-promoting agent, and an antigenicity-promoting agent. In certain embodiments, the agent is a protein or a peptide expressed by the dsDNA molecule.

In certain embodiments, the integration-promoting agent is selected from the group consisting of INS peptide (WTAVQMAVFIHNFKRK; SEQ ID NO:1), INr1 peptide (WTHLEGKIILVAVHVA; SEQ ID NO:2), INr2 peptide (WGSNFTSTTVKA; SEQ ID NO:3), and LEDGF/p75 protein (UniProt O75475), a peptide comprising SEQ ID NO:4, a peptide comprising SEQ ID NO:5, and a peptide comprising SEQ ID NO:6, and any combination thereof. Each possibility represents a separate embodiment of the invention. In certain embodiments, the integration-promoting agent is a combination of at least two of the peptides: INS (WTAVQMAVFIHNFKRK; SEQ ID NO:1), INr1 (WTHLEGKIILVAVHVA; SEQ ID NO:2), and INr2 (WGSNFTSTTVKA; SEQ ID NO:3).

In certain embodiments, the apoptosis-promoting agent is selected from the group consisting of caspase 3 (UniProt P42574) protein and DNA-dependent protein kinase (DNA-PK) (UniProt P78527) protein. In certain embodiments, the antigenicity-promoting agent is selected from the group consisting of a cancer-associated antigen and a pathogen-associated antigen.

In certain embodiments, the antigenicity-promoting agent reactivates a latent or dormant pathogen to express a pathogen-associated antigen.

In certain embodiments, the latent or dormant pathogen is an integrated HIV provirus.

In certain embodiments, the antigenicity-promoting agent is selected from the group consisting of inhibitors of histone deacetylase (HDAC), suberoylanilide hydroxamic acid (SAHA), Ro5-3335 (CAS number 30195-30-3), vorinostat (CAS number 149647-78-9), panobinostat (CAS number 404950-80-7), protein kinase C (PKC) activators, prostratin (CAS number 60857-08-1) and bryostatin (CAS number 83314-01-6).

The present invention thus provides, according to some embodiments, a method of treating cancer comprising administering to a subject in need thereof, a complex comprising (i) a linear molecule of double-stranded DNA (dsDNA) comprising long term repeat (LTR) sequences recognized by the integrating enzyme of (ii), (ii) an integrating enzyme, capable of entering the nuclei of cells after binding to the LTR sequences of the dsDNA molecule of (i) and creating multiple double strand breaks (DSBs) in the chromosomal DNA of the cells, and (iii) a targeting moiety capable of binding at least one molecule presented by the cancer cells, and (b) administering to the subject at least one integration-promoting agent.

According to some embodiments, the complex is a lentivirus particle comprising the linear dsDNA molecule, an integrase, and an antibody directed against a cell surface marker.

According to some embodiments, the cell surface marker is selected from CD24 and CD20.

According to a specific embodiment, a method of treating CD24 expressing cancer is provided comprising administering to a subject in need thereof, a complex comprising (i) a lentivirus particle comprising a linear molecule of double-stranded DNA (dsDNA) comprising long term repeat (LTR) sequences recognized by the integrating enzyme of (ii), (ii) an integrase, capable of entering the nuclei of cells after binding to the LTR sequences of the dsDNA molecule of (i) and creating multiple double strand breaks (DSBs) in the chromosomal DNA of the cells, and (iii) an antibody capable of binding CD24 presented by the cancer cells, and (b) administering to the subject at least one integration-promoting agent selected from the group consisting of: SEQ ID Nos 1-6, and any combination thereof. Each possibility represents a separate embodiment of the invention.

The present invention further provides, in another aspect, a method for promoting DSBs in the chromosomal DNA of a cell, comprising the steps of introducing to the cell a molecule of dsDNA having at least one strand capable of specific binding to an integrating enzyme, and a dsDNA molecule, and an integrating enzyme, capable of specific binding to the molecule of dsDNA, entering the nucleus of the cell, and creating DSBs in the chromosomal DNA of the cell.

The present invention further provides, in another aspect, a method for promoting DSBs in the chromosomal DNA of a cell, comprising the steps of introducing to the cell (a) a complex comprising: (i) a linear molecule of dsDNA comprising long term repeat (LTR) sequences recognized by the integrating enzyme of (ii), (ii) an integrating enzyme, capable of entering the nuclei of the cells after binding to the LTR sequences of the dsDNA molecule of (i) and creating multiple double strand breaks (DSBs) in the chromosomal DNA of the cells, and (iii) a targeting moiety capable of binding specifically to a molecule presented by the cell; and (b) at least one integration-promoting agent.

In certain embodiments of the methods described above, the cell is in the body of a human subject.

In certain embodiments, any of the methods described above comprises contacting a cancer cell with (i) a molecule of double-stranded DNA (dsDNA), (ii) an integrating enzyme, and (iii) an integration-promoting agent selected from the group consisting of INS peptide (WTAVQMAVFIHNFKRK; SEQ ID NO:1), INr1 peptide (WTHLEGKIILVAVHVA; SEQ ID NO:2), INr2 peptide (WGSNFTSTTVKA; SEQ ID NO:3), LEDGF/p75 protein (UniProt O75475), and any combination thereof. Each possibility represents a separate embodiment of the invention.

In certain embodiments, the integration-promoting agent is a combination of at least two of: INS peptide, INr1 peptide, and INr2 peptide.

The present invention further provides, in another aspect, a pharmaceutical composition, comprising a molecule of dsDNA having at least one strand capable of specific binding to an integrating enzyme, and an integrating enzyme, capable of specific binding to the molecule of dsDNA, entering the nucleus of a cell, and creating DSBs in the chromosomal DNA of the cell.

The present invention further provides, in another aspect, a pharmaceutical composition, comprising (i) a linear molecule of dsDNA comprising long term repeat (LTR) sequences recognized by the integrating enzyme of (ii), and (ii) an integrating enzyme, capable of entering the nuclei of cells after binding to the LTR sequences of the dsDNA molecule of (i) and creating multiple DSBs in the chromosomal DNA of the cells.

According to some embodiments, the pharmaceutical composition comprises:
(i) a linear molecule of dsDNA comprising long term repeat (LTR) sequences recognized by the integrating enzyme of (ii), and
(ii) an integrating enzyme, capable of entering the nuclei of cells after binding to the LTR sequences of the dsDNA molecule of (i) and creating multiple double strand breaks (DSBs) in the chromosomal DNA of the cells, or a polynucleotide sequence encoding said integrating enzyme; and
(iii) an integration-promoting agent selected from the group consisting of: INS peptide (WTAVQMAVFIHNFKRK; SEQ ID NO:1), INr1 peptide (WTHLEGKIILVAVHVA; SEQ ID NO:2), INr2 peptide (WGSNFTSTTVKA; SEQ ID NO:3), LEDGF/p75 protein (UniProt O75475), and any combination thereof, or a polynucleotide sequence encoding said integration-promoting agent.

According to some embodiments, the linear molecule of dsDNA encodes the integrating enzyme and/or the integration-promoting agent.

In certain embodiments, the pharmaceutical composition further comprises at least one integration-promoting agent selected from the group consisting of: INS peptide (WTAVQMAVFIHNFKRK; SEQ ID NO:1), INr1 peptide (WTHLEGKIILVAVHVA; SEQ ID NO:2), INr2 peptide (WGSNFTSTTVKA; SEQ ID NO:3), LEDGF/p75 protein (UniProt O75475), a peptide comprising SEQ ID NO:4, a peptide comprising SEQ ID NO:5, and a peptide comprising SEQ ID NO:6, and any combination thereof. Each possibility represents a separate embodiment of the invention. In certain embodiments, the integration-promoting agent is a combination of at least two of: INS peptide, INr1 peptide, and INr2 peptide.

In certain embodiments, the pharmaceutical composition comprises, a complex comprising (i) a linear molecule of double-stranded DNA (dsDNA) comprising long term repeat (LTR) sequences recognized by the integrating enzyme of (ii), (ii) an integrating enzyme, capable of entering the nuclei of cells after binding to the LTR sequences of the dsDNA molecule of (i) and creating multiple double strand breaks (DSBs) in the chromosomal DNA of the cells, and (iii) a targeting moiety capable of binding at least one molecule presented by the cancer cells.

According to some embodiments, the complex is a lentivirus particle comprising the linear dsDNA molecule, an integrase, and an antibody directed against a cell surface marker.

According to some embodiments, the cell surface marker is selected from CD24 and CD20.

According to a specific embodiment, the pharmaceutical composition comprises, a complex comprising (i) a lentivirus particle comprising a linear molecule of double-stranded DNA (dsDNA) comprising long term repeat (LTR) sequences recognized by the integrating enzyme of (ii), (ii) an integrase, capable of entering the nuclei of cells after binding to the LTR sequences of the dsDNA molecule of (i) and creating multiple double strand breaks (DSBs) in the chromosomal DNA of the cells, and (iii) an antibody capable of binding CD24 presented by the cancer cells.

According to some embodiments, the pharmaceutical composition further comprises at least one integration-promoting agent selected from the group consisting of: SEQ ID Nos 1-6, and any combination thereof. Each possibility represents a separate embodiment of the invention.

The at least one integration-promoting agent may be contained in the pharmaceutical composition as a peptide or as polynucleotide sequence encoding at least one peptide.

The present invention further provides, in another aspect, a kit, comprising a composition comprising a molecule of dsDNA having at least one strand capable of specific binding to an integrating enzyme, and a dsDNA molecule, and a composition comprising an integrating enzyme, capable of specific binding to the dsDNA molecule, entering the nucleus of a cell, and creating DSBs in the chromosomal DNA of the cell.

The present invention further provides, in another aspect, a kit, comprising (i) a composition comprising a linear molecule of double-stranded DNA (dsDNA) comprising long term repeat (LTR) sequences recognized by the integrating enzyme of (ii), and (ii) a composition comprising an integrating enzyme, capable of entering the nuclei of cells after binding to the LTR sequences of the dsDNA molecule of (i) and creating multiple DSBs in the chromosomal DNA of the cells.

In certain embodiments, the kit further comprises a composition comprising at least one integration-promoting agent selected from the group consisting of: INS peptide (WTAVQMAVFIHNFKRK; SEQ ID NO:1), INr1 peptide (WTHLEGKIILVAVHVA; SEQ ID NO:2), INr2 peptide (WGSNFTSTTVKA; SEQ ID NO:3), LEDGF/p75 protein (UniProt O75475), a peptide comprising SEQ ID NO:4, a peptide comprising SEQ ID NO:5, and a peptide comprising SEQ ID NO:6, and any combination thereof. Each possibility represents a separate embodiment of the invention. In certain embodiments, the integration-promoting agent is a combination of at least two of: INS peptide, INr1 peptide, and INr2 peptide.

In certain embodiments, the pharmaceutical compositions described above, or the kits described above, are for use in promoting DSBs in the chromosomal DNA of a specific population of cells.

In certain embodiments, the pharmaceutical compositions described above, or the kits described above, are for use in treating a disease or condition selected from the group consisting of cancer, viral-infection, obesity, yeast infection and bacterial infection.

In certain embodiments, the specific population of cells is selected from the group consisting of cancer cells, virally-infected cells, lipocytes, yeast cells and bacteria cells.

Other objects, features and advantages of the present invention will become clear from the following description.

### BRIEF DESCRIPTION OF THE DRAWINGS

FIG. 1A is light and florescence microscope images of breast cancer cells (BT549) infected with LV-scFvCD24 particles according to certain embodiments the present invention, as described in Example 1.

FIG. 1B is light and florescence microscope images of lung cancer cells (H1975) infected with LV-scFvCD24 particles according to certain embodiments the present invention, as described in Example 1.

FIG. 2A is a schematic representation of the order of different steps in the experiment described in Example 2.

FIG. 2B is a bar graph depicting the survival rate of the lung cancer cells (H1975) in the experiment described in Example 2.

FIG. 3A is graph depicting the FITC signal from uninfected and infected lung cancer cells (H1975) in Example 3.

FIG. 3B is a bar graph depicting the survival rate of the lung cancer cells (H1975) in the experiment described in Example 3.

FIG. 3C is a schematic representation of the order of different steps in the experiment described in Example 3.

FIG. 4A is light and florescence microscope images of colorectal adenocarcinoma cancer cells (DLD1) infected with LV-scFvCD24 particles according to certain embodiments the present invention, as described in Example 4.

FIG. 4B is light and florescence microscope images of breast adenocarcinoma cancer cells (MCF7) infected with LV-scFvCD24 particles according to certain embodiments the present invention, as described in Example 4.

FIG. 5A is a bar graph depicting the relative activity of integrase enzyme with and without INS peptide in the experiment described in Example 5.

FIG. 5B is a schematic representation of the order of different steps in the experiment described in Example 5.

FIG. 5C is a bar graph depicting the survival rate of lung cancer cells (H1975) in the experiment described in Example 5.

FIG. 5D is a bar graph depicting the survival rate of breast cancer cells (BT549) in the experiment described in Example 5.

FIG. 6A is light and florescence microscope images of lung cancer cells (H1975) infected with particles according to certain embodiments the present invention, as described in Example 6.

FIG. 6B is a schematic representation of the order of different steps in the experiment described in Example 6.

FIG. 6C is a bar graph depicting the survival rate of pancreatic carcinoma cells (Panc-1) in the experiment described in Example 6.

FIG. 6D is a bar graph depicting the survival rate of non-small-cell lung cancer cells (H1975) in the experiment described in Example 6.

FIG. 7A is a line graph depicting the change in volume over time of H1975 tumors in the experiment described in Example 7.

FIG. 7B is a picture of certain organs removed at the end of the experiment described in Example 7.

FIG. 7C is a picture of the fluorescence emitted from certain organs removed at the end of the experiment described in Example 7.

FIG. 8 is a schematic representation of the order of different steps in the experiment described in Example 9.

### DETAILED DESCRIPTION OF THE INVENTION

The present invention provides methods and means for the safe, highly specific eradication of substantially any type of an undesired cell, by utilizing the cell's unique expression profile or external markers, which distinguishes the cell from normal, healthy or otherwise favorable cells. The present invention provides a combinatorial solution to the problem of attaining both specificity and efficacy in fighting an array of human diseases and conditions. Specifically, the present invention provides a system for inflicting destructive DNA damage levels by specific targeting of DNA-impairing retroviral integrase enzymes to the nuclei of target cells.

Without being bound to any theory or mechanism, the present invention provides a modular system, made of at least two elements. The first element is an enzyme capable of inflicting DNA damage, in a rate which would outpace the natural DNA repair capabilities found in all cells. Prominent examples of such an enzyme are integrase enzymes, found in the virions of all retroviruses. A retrovirus genome is a single-stranded positive sense RNA virus with a DNA intermediate. Once inside the host cell cytoplasm, the virus uses its own reverse transcriptase enzyme to produce DNA from its RNA genome. This new DNA is then incorporated into the host cell genome by an integrase enzyme, which catalyzes a concerted cleavage and joining reaction in which the 3′ ends of the viral DNA are joined to the 5′ ends of a double-stranded break in the host DNA. The present invention exploits this integrase capability of catalyzing double-strand DNA breaks to inflict toxic levels of DNA breaks. The second element is a double-strand DNA molecule, which “mimics” the retro-transcribed RNA molecule found in retroviruses, by having 5′ and 3′ ends which are similar or identical in length or sequence to the long terminal repeats (LTRs) found in the double-strand DNA molecule retro-transcribed from the RNA genome of retroviruses. The LTR sequences are recognized by the integrase enzyme thereby forming a complex, which is then translocated to the cell's nucleus. The present invention exploits the capability of the double-strand DNA molecule to bind to the integrase enzyme and transfer it to the cell nucleus, thereby bringing the integrase enzyme into proximity with the chromosomal DNA of the cell.

The first step of the integration process occurs in the cytoplasm of the host cell following the completion of reverse transcription of the HIV RNA into complementary DNA (c-DNA). This step involves the binding of integrase—most likely in the dimer form—to each end of the newly formed HIV c-DNA. The binding takes place at specific sequences in the long terminal repeat (LTR) regions. The integrase-HIV DNA complex is part of an intracellular nucleoprotein particle known as the “pre-integration complex” (PIC). This complex consists of linear HIV DNA, viral proteins, and host proteins. The viral proteins include integrase, nucleocapsid, matrix, viral protein R (Vpr), and reverse transcriptase. Several host proteins can also form part of this complex, although it is unclear whether some or all join the pre-integration complex prior to nuclear transport.

In the second step of the integration process, which also takes place in the host cytoplasm, the integrase dimer cleaves the viral DNA at each 3′ end. This cleavage reaction removes GT di-nucleotides on the 3′-side of a conserved CA dinucleotide region. The cleavage of the dinucleotide at each viral DNA 3′-end generates a dinucleotide 5′ “overhang” and a reactive intermediate that contains a 3′-hydroxyl group. This 3′ processing step is the first of two key catalytic reactions performed by the integrase enzyme, and it prepares the viral DNA for integration into the host DNA. In an alternative view of the DNA binding and 3′-processing reaction, the tetramer form of integrase (not the dimer) binds to the ends of the HIV DNA, and then cleaves the 3′ ends.

In the third step of the integration process, the pre-integration complex is transported into the nucleus of the host cell, entering through one of the nuclear pore complexes.

Inside the nucleus, the host protein lens epithelium-derived growth factor/p75, commonly referred to in abbreviated form as LEDGF/p75, binds to the pre-integration complex and the host DNA. The LEDGF/p75 (also known as PSIP1, DFS70, LEDGF, PAIP, PSIP2, p52, p75, UniProt ID O75475) serves as a tethering protein (or bridge) between the pre-integration complex and the host DNA. The sequence of binding of the LEDGF/p75, the host DNA, and the pre-integration complex remains unclear. In one version, the LEDGF/p75 binds first to the pre-integration complex and then to the host DNA. On the other hand, LEDGF/p75 may bind first to the host DNA and then to the pre-integration complex. Regardless of the sequence, it is believed that the presence of LEDGF/p75 results in the integrase dimers approaching each other to form a tetramer.

The next step, the strand transfer reaction, takes place inside the host cell nucleus and involves the critical step of inserting the HIV DNA into a selected region of the host DNA. The region of insertion contains a weakly conserved palindromic sequence. This strand transfer reaction is initiated as the HIV integrase catalyzes the HIV DNA 3′-hydroxyl group attack on the host DNA. The attack by the HIV DNA occurs on opposite strands of the host DNA in a staggered fashion, typically 4-6 base pairs apart. This reaction leads to separation of the bonds in the host DNA base pairs located between the staggered cuts, and the joining of the HIV 3′-hydroxyl groups with the host DNA 5′ phosphate ends. At this point, the newly joined viral-host DNA region unfolds.

Following the strand transfer process, the HIV-DNA and host DNA junctions have unpaired regions of DNA, referred to as DNA “gaps”. In addition, the two base pairs at the end of the 5′ region of the viral DNA remain unpaired after the strand transfer. The insertion of the new HIV DNA and the remaining gaps that flank the integration site induce a host cellular DNA damage response. The host response is critical in the final step of integration, known as “gap repair”. The gap repair requires at least three host enzymes—polymerase, nuclease, and ligase. In the first step of gap repair, the polymerase enzymes extend the host DNA on each end and, thus, fill in the gaps. Next, nuclease enzymes remove the 5′ dinucleotide “flaps” on the HIV DNA. Last, the DNA ligase enzymes join the remaining unbound segment of the HIV and host DNA strands. This gap repair process completes the integration of the HIV DNA into the host DNA, with the fully integrated HIV DNA now being referred to as “pro-viral DNA”.

The present invention therefore provides, in one aspect, a method for selectively destroying a population of target cells, comprising the steps of contacting the cells with a molecule of dsDNA having at least one strand capable of specific binding to the integrating enzyme, and a dsDNA molecule, and an integrating enzyme, capable of specific binding to the dsDNA molecule, entering the nucleus of a cell, and creating DSBs in the chromosomal DNA of the cell, wherein the target cell is selected from the group consisting of a cancer cell, a virally-infected cell, a lipocyte, a yeast cell and a bacteria cell, and wherein the dsDNA molecule, the integrating enzyme, or both, are selectively targeted to the cell

The present invention further provides, in another aspect, a method for treating a disease or condition in a subject in need of such treatment, comprising the steps of administering to the subject a molecule of dsDNA having at least one strand capable of specific binding to an integrating enzyme, and a dsDNA molecule, and an integrating enzyme, capable of specific binding to the dsDNA molecule, entering the nucleus of a cell, and creating DSBs in the chromosomal DNA of the cell.

In certain embodiments, the disease or condition is selected from the group consisting of cancer, viral-infection, obesity, yeast infection and bacterial infection, and wherein the dsDNA molecule, the integrating enzyme, or both, are selectively targeted to the cell.

The term “method” as used herein generally refers to manners, means, techniques and procedures for accomplishing a given task, including, but not limited to, those manners, means, techniques and procedures either known to, or readily developed from known manners, means, techniques and procedures by practitioners of the chemical, pharmacological, biological, biochemical and medical arts.

The term “treating” as used herein includes the diminishment, alleviation, or amelioration of at least one symptom associated or induced by a disease or condition. The term “treating” as used herein also includes preventative (e.g., prophylactic), palliative and curative treatment.

The term “cancer” as used herein refers to refers to one of a group of diseases caused by the uncontrolled, abnormal growth of cells, which optionally may spread to adjoining tissues or other parts of the body.

The terms “viral infection”, “yeast infection” and “bacterial infection” as used herein refers to any stage of a viral, yeast or bacterial infection, including incubation phase, latent or dormant phase, acute phase, and development and maintenance of immunity towards a virus, yeast or bacteria. The terms also include any clinical sign, symptom or disease that occurs in an animal or human subject following contamination of the animal or human subject by a virus, yeast or bacteria. Accordingly, these terms include both contamination by the virus, yeast or bacteria, and the various pathologies which are the consequence of contamination by the virus, yeast or bacteria.

Obesity increases the likelihood of various diseases, particularly heart disease, type 2 diabetes, obstructive sleep apnea, certain types of cancer, and osteoarthritis. Obesity is most commonly caused by a combination of excessive food energy intake, lack of physical activity, and genetic susceptibility, although a few cases are caused primarily by genes, endocrine disorders, medications, or psychiatric illness. Evidence to support the view that some obese people eat little yet gain weight due to a slow metabolism is limited. On average, obese people have greater energy expenditure than their thin counterparts due to the energy required to maintain an increased body mass. Adipocytes, also known as lipocytes and fat cells, are the cells that primarily compose adipose tissue, specialized in storing energy as fat. There are two types of adipose tissue, white adipose tissue (WAT) and brown adipose tissue (BAT), which are also known as white fat and brown fat, respectively, and comprise two types of fat cells.

The term “obesity” as used herein refers to a medical condition in which excess body fat has accumulated to the extent that it may have a negative effect on health, leading to reduced life expectancy and/or increased health problems.

The phrase “having at least one strand capable of specific binding to an integrating enzyme” as used herein refers to a DNA strand having a sequence, e.g. a 3′ sequence, such as found at the 3′ ends of both strands of a double stranded DNA molecule which was reverse-transcribed by a retroviral reverse-transcriptase (RT) enzyme from a retroviral RNA molecule. Retroviral integrase (IN) enzyme carries out vDNA integration following two consecutive steps: 3′-P processing in the cytoplasm and strand transfer (ST) in the nucleus. For 3′-P, IN processes vDNA by cleaving its 3′-end immediately after a conserved CA dinucleotide, thereby releasing a GT dinucleotide from each long terminal repeats (LTRs) 3′-ends.

The term “retrovirus-derived vector” as used herein refers to an infective one-cycle particle, comprising an RNA molecule, a reverse-transcriptase (RT) enzyme and integrase (IN) enzyme, which are encapsulated by a membrane.

The term “integrating enzyme” as used herein refers to any enzyme capable of creating double stranded breaks (DSBs) in the chromosomal DNA of a human cell. The “integrating enzyme” may optionally be further capable of incorporating a double stranded DNA molecule into the gap formed by the DSBs. Non-limiting examples of integrating enzymes are the integrase enzymes of retroviruses, such as the integrase enzyme of HIV-1.

In certain embodiments, the term “HIV-1 integrase” as used herein relates e.g. to the protein having the UniProtKB Entry Q76353 (Q76353_9HIV1). In certain embodiments, the term “HIV-2 integrase” as used herein relates e.g. to the protein having the UniProtKB Entry D5LQ24 (D5LQ24_9HIV2). Due to the high sequence diversity in HIV genes, many other variants of HIV-1 integrase and HIV-2 integrase are further considered as “integrating enzymes” according to the present invention.

The phrase “capable of specific binding to the dsDNA molecule, entering the nucleus of a cell, and creating DSBs in the chromosomal DNA of the cell” as used herein refers to any integrating enzyme which specifically recognizes the LTR-like sequences in at least one end of a dsDNA molecule, forms a complex with the dsDNA molecule, optionally involving other proteins, moves into the nucleus of the cell in which it is present, and creates a double stranded break in the chromosomal DNA of the cell.

Any linear dsDNA sequence comprising LTR sequences recognized by the integrating enzyme, can be used according to the present invention. dsDNA sequences according to the present invention may be naturally occurring, or non-naturally occurring molecules, and include isolated sequences, random sequences and synthetically produced sequences, or combinations thereof. Any method known in the art to isolate or synthesize dsDNA sequences may be used according to the present invention. In certain embodiments, the dsDNA molecule is a synthetic dsDNA molecule that has been prepared entirely or at least partially by chemical means. Synthetic DNA sequences may be used, for example, for modifying native DNA sequences in terms of codon usage and expression efficiency.

Cancers are a large family of diseases that involve abnormal cell growth with the potential to invade or spread to other parts of the body. They form a subset of neoplasms. A neoplasm or tumor is a group of cells that have undergone unregulated growth, and will often form a mass or lump, but may be distributed diffusely. Six characteristics of cancer have been proposed: self-sufficiency in growth signaling, insensitivity to anti-growth signals, evasion of apoptosis, enabling of a limitless replicative potential, induction and sustainment of angiogenesis, and activation of metastasis and invasion of tissue. The progression from normal cells to cells that can form a discernible mass to outright cancer involves multiple steps known as malignant progression. In certain embodiments, the disease or condition is cancer.

Long terminal repeats (LTRs) are identical sequences of DNA that repeat hundreds or thousands of times found at either end of proviral DNA formed by reverse transcription of retroviral RNA. They are used by viruses to insert their genetic material into the host genomes. The LTRs are partially transcribed into an RNA intermediate, followed by reverse transcription into complementary DNA (cDNA) and ultimately dsDNA (double-stranded DNA) with full LTRs. The LTRs then mediate integration of the retroviral DNA via an LTR specific integrase into another region of the host chromosome. The multi-step process of reverse transcription results in the placement of two identical LTRs, at either end of the proviral DNA. The ends of the LTRs subsequently participate in integration of the provirus into the host genome. Retroviral integrase catalyzes 3′-processing, in which two or three nucleotides are removed from one or both 3′ ends of the viral DNA to expose the invariant CA dinucleotide at both 3′-ends of the viral DNA. In certain embodiments, both strands of the dsDNA molecule are capable of specific binding to the integrating enzyme. In certain embodiments, both strands of the dsDNA molecule comprise an LTR sequence recognized by the integrating enzyme. In certain embodiments, both strands of the dsDNA molecule comprise a cytosine-adenine dinucleotide (CA) near their 3′ end. In certain embodiments, both strands of the dsDNA molecule comprise a cytosine-adenine dinucleotide at a distance of 2-20 nucleotides 5′ to their 3′ end. In certain embodiments, both strands of the dsDNA molecule comprise a cytosine-adenine dinucleotide at a distance of 1, 2, 3, 4 or 5 nucleotides 5′ to their 3′ end. Each possibility represents a separate embodiment of the invention.

Generally, vectors derived from retroviruses comprise an RNA molecule, a reverse-transcriptase (RT) enzyme and an integrase (IN) enzyme, as well as some other viral proteins, all enveloped by a membrane. The RNA molecule are reverse-transcribed by the RT enzyme to a dsDNA molecule, which would be incorporated into the chromosomal DNA of the infected host by the IN enzyme, wherein both ends of the RNA molecule are reverse-transcribed by the RT enzyme to the long terminal repeats described above. In certain embodiments, the dsDNA molecule is reverse-transcribed from a retrovirus-derived RNA molecule. In certain embodiments, the RNA molecule is part of a retrovirus-derived vector. In certain embodiments, the retrovirus-derived vector is specifically targeted to the cell by comprising a targeting agent, capable of specific binding to a target molecule presented by the cell. In certain embodiments, the targeting agent is an antibody capable of specific binding to an antigen presented by the cell. In certain embodiments, the retrovirus-derived vector further comprises the integrating enzyme and a reverse-transcriptase enzyme.

As an alternative to the use of retrovirus and lentivirus vectors for the delivery of the dsDNA molecule to the target cell, synthetic dsDNA molecules may be readily produced by standard, well-known techniques. In certain embodiments, the dsDNA molecule is a synthetic dsDNA molecule. In certain embodiments, the synthetic dsDNA molecule is naked or encapsulated by a shell. In certain embodiments, the shell comprises a targeting agent, capable of specific binding to a target molecule presented by the cell. In certain embodiments, the targeting agent is an antibody capable of specific binding to an antigen presented by the cell. In certain embodiments, the shell further encapsulates the integrating enzyme.

The term “targeting agent” as used herein refers to molecule or compound that binds a target found, expressed or presented by a target cell.

The term “shell” as used herein refers to any material encapsulating any type of content. Non-limiting examples of shells are lipid membranes such as lipid bilayer membranes, single-layer and multi-lamellar liposomes. Other non-limiting examples of shells are viruses and virus-derived infective vectors.

Retroviral integrase is an enzyme produced by a retrovirus (such as HIV) that enables its genetic material to be integrated into the DNA of the infected cell. Since retroviruses are rapidly and constantly changing, the exact sequence of retroviral integrase is practically impossible to follow. In certain embodiments, the integrating enzyme is an integrase enzyme selected from the group consisting of HIV-1 integrase, HIV-2 integrase, active fragments thereof, and active analogs thereof. Each possibility represents a separate embodiment of the invention.

The term “active fragment” as used herein refers to any fragment of consecutive amino-acids found in a retroviral integrase which maintains at least 25%, at least 50%, at least 75% or at least 90% of the biological activities of the retroviral integrase.

The term “active analog” as used herein refers to any protein, polypeptide or peptide which demonstrates at least 25%, at least 50%, at least 75% or at least 90% of the biological activities of the retroviral integrase.

In certain embodiments, the integrating enzyme is part of a retrovirus-derived vector. In certain embodiments, the retrovirus-derived vector is specifically targeted to the cell by comprising a targeting agent, capable of specific binding to a target molecule presented by the cell. In certain embodiments, the targeting agent is an antibody capable of specific binding to an antigen presented by the cell. In certain embodiments, the retrovirus-derived vector further comprises the dsDNA molecule.

In certain embodiments, the integrating enzyme is encapsulated by a shell. In certain embodiments, the shell comprises a targeting agent, capable of specific binding to a target molecule presented by the cell. In certain embodiments, the targeting agent is an antibody capable of specific binding to an antigen presented by the cell. In certain embodiments, the shell further encapsulates the dsDNA molecule.

In certain embodiments, the method described above further comprises the step of introducing to the cell at least one agent selected from the group consisting of an integration-promoting agent, an apoptosis-promoting agent, and an antigenicity-promoting agent. In certain embodiments, the agent is a protein or a peptide expressed by the dsDNA molecule.

The term “integration-promoting agent” as used herein refers to any molecule or compound which would increase the number of integration events within a cell.

The term “apoptosis-promoting agent” as used herein refers to any molecule or compound which would initiate or promote an apoptotic cascade within a cell.

The term “antigenicity-promoting agent” as used herein refers to any molecule or compound which would increase the antigenicity of a cell towards the immune system of the subject.

In certain embodiments, the integration-promoting agent is selected from the group consisting of INS (WTAVQMAVFIHNFKRK; SEQ ID NO:1) peptide, INr1 (WTHLEGKIILVAVHVA; SEQ ID NO:2) peptide, INr2 (WGSNFTSTTVKA; SEQ ID NO:3) peptide, LEDGF/p75 (UniProt O75475) protein, a peptide comprising SEQ ID NO:4, a peptide comprising SEQ ID NO:5, and a peptide comprising SEQ ID NO:6, and any combination thereof. In certain embodiments, the integration-promoting agent is a combination of INS (WTAVQMAVFIHNFKRK; SEQ ID NO:1) peptide, INr1 (WTHLEGKIILVAVHVA; SEQ ID NO:2) peptide, and INr2 (WGSNFTSTTVKA; SEQ ID NO:3) peptide. In certain embodiments, the apoptosis-promoting agent is selected from the group consisting of caspase 3 (UniProt P42574) protein and DNA-dependent protein kinase (DNA-PK) (UniProt P78527) protein. In certain embodiments, the antigenicity-promoting agent is selected from the group consisting of a cancer-associated antigen and a pathogen-associated antigen. In certain embodiments, the antigenicity-promoting agent reactivates a latent or dormant pathogen to express a pathogen-associated antigen. In certain embodiments, the latent or dormant pathogen is an integrated HIV provirus, and the antigenicity-promoting agent is selected from the group consisting of inhibitors of histone deacetylase (HDAC), suberoylanilide hydroxamic acid (SAHA), Ro5-3335 (CAS number 30195-30-3), vorinostat (CAS number 149647-78-9), panobinostat (CAS number 404950-80-7), protein kinase C (PKC) activators, prostratin (CAS number 60857-08-1) and bryostatin (CAS number 83314-01-6).

In certain embodiments, the cell is selected from the group consisting of a cancer cell, a virally-infected cell, a lipocyte, a yeast cell and a bacteria cell.

The present invention further provides, in another aspect, a method for promoting double-stranded DNA breaks (DSBs) in the chromosomal DNA of a cell, comprising the steps of introducing to the cell a molecule of double-stranded DNA (dsDNA) having at least one strand capable of specific binding to an integrating enzyme, and an integrating enzyme, capable of specific binding to the molecule of dsDNA, entering the nucleus of the cell, and creating DSBs in the chromosomal DNA of the cell.

It is important to note that while the methods described above may take place or be performed ex-vivo, e.g. on grafts, cells or tissues removed from the body of a subject, these methods are highly suited to be performed in-vivo. In certain embodiments of the methods described above, the cell is in the body of a human subject.

As an alternative to the selective targeting of the dsDNA molecule, the integrating enzyme, or both, to the target cells, or as an added control or safety feature, the delivery of the integrating enzyme to the target cells may be achieved by selective expression of the integrating enzyme in the cells. Selective expression of transgenes may be accomplished, for example, by placing the expression of the integrating enzyme under the control of a selective promoter, i.e. a promoter which allows expression of its adjacent transgene in predefined circumstances. For example, when treating cancer, the use of promoters of cancer-associated proteins is advisable for expression of the integrating enzyme. Alternatively, when treating pathogen infection, the use of promoters of pathogen-associated proteins is advisable for expression of the integrating enzyme. Yet alternatively, when treating obesity, the use of promoters of adipocyte-associated proteins is advisable for expression of the integrating enzyme. In certain embodiment, the integrating enzyme is expressed in the cell under the control of a promoter of a cancer-associated protein. In certain embodiment, the integrating enzyme is expressed in the cell under the control of a promoter of a pathogen-associated protein. In certain embodiment, the integrating enzyme is expressed in the cell under the control of a promoter of an adipocyte-associated protein.

The present invention further provides, in another aspect, a pharmaceutical composition, comprising a molecule of dsDNA having at least one strand capable of specific binding to an integrating enzyme, and an integrating enzyme, capable of specific binding to the molecule of dsDNA, entering the nucleus of a cell, and creating double-stranded DNA breaks (DSBs) in the chromosomal DNA of the cell.

The term “pharmaceutical composition” as used herein refers to any composition comprising at least one biologically active agent, and at least one pharmaceutically acceptable carrier. Non-limiting example of biologically active molecules are an integrase enzyme and a dsDNA molecule capable of specific binding to this enzyme. The term “agent” as used herein refers to any molecule having a biological activity or function.

As used herein, the term “pharmaceutically acceptable carrier” means a pharmaceutically acceptable material, composition or vehicle, such as a liquid or solid filler, diluent, excipient, solvent or encapsulating material, involved in carrying or transporting a compound(s) of the present invention within or to the subject such that it can performs its intended function. A carrier must be “acceptable” in the sense of being compatible with the other ingredients of the formulation and not injurious to the subject.

The present invention further provides, in another aspect, a kit, comprising a composition comprising a molecule of dsDNA having at least one strand capable of specific binding to an integrating enzyme, and a composition comprising an integrating enzyme, capable of specific binding to the dsDNA molecule, entering the nucleus of a cell, and creating DSBs in the chromosomal DNA of the cell.

In certain embodiments, the pharmaceutical compositions described above are for use in promoting DSBs in the chromosomal DNA of a cell. In certain embodiments, the pharmaceutical compositions described above are for use in treating a disease or condition in a subject in need of such treatment, wherein the disease or condition is selected from the group consisting of cancer, viral-infection, obesity, yeast infection and bacterial infection. In certain embodiments of the pharmaceutical compositions described above, the cell is selected from the group consisting of a cancer cell, a virally-infected cell, a lipocyte, a yeast cell and a bacteria cell.

In certain embodiments, the kits described above are for use in promoting DSBs in the chromosomal DNA of a cell. In certain embodiments, the kits described above are for use in treating a disease or condition in a subject in need of such treatment, wherein the disease or condition is selected from the group consisting of cancer, viral-infection, obesity, yeast infection and bacterial infection. In certain embodiments of the kits described above, the cell is selected from the group consisting of a cancer cell, a virally-infected cell, a lipocyte, a yeast cell and a bacteria cell.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

The present invention further provides, in another aspect, a composition comprising (i) a linear molecule of double-stranded DNA (dsDNA) comprising long term repeat (LTR) sequences recognized by the integrating enzyme of (ii), and (ii) an integrating enzyme, capable of entering the nuclei of the cells after binding to the LTR sequences of the dsDNA molecule of (i) and creating multiple double strand breaks (DSBs) in the chromosomal DNA of cells.

In certain embodiments, the composition further comprises (iii) a targeting moiety capable of binding the composition specifically to a molecule presented by a cell, or (iv) an integration-promoting agent selected from the group consisting of INS peptide (WTAVQMAVFIHNFKRK; SEQ ID NO:1), INr2 peptide (WGSNFTSTTVKA; SEQ ID NO:3), INr1 peptide (WTHLEGKIILVAVHVA; SEQ ID NO:2), LEDGF/p75 protein (UniProt O75475), a peptide comprising SEQ ID NO:4, a peptide comprising SEQ ID NO:5, and a peptide comprising SEQ ID NO:6, and any combination thereof; or both (iii) and (iv). In certain embodiments, the integration-promoting agent is a combination of at least two of: INS peptide, INr1 peptide, and INr2 peptide.

Various embodiments and aspects of the present invention as delineated hereinabove and as claimed in the claims section below find experimental support in the following examples.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1. In-Vitro Evaluation of Infection LV-scFvCD24 Particles in Various Human Cancer Cell Lines

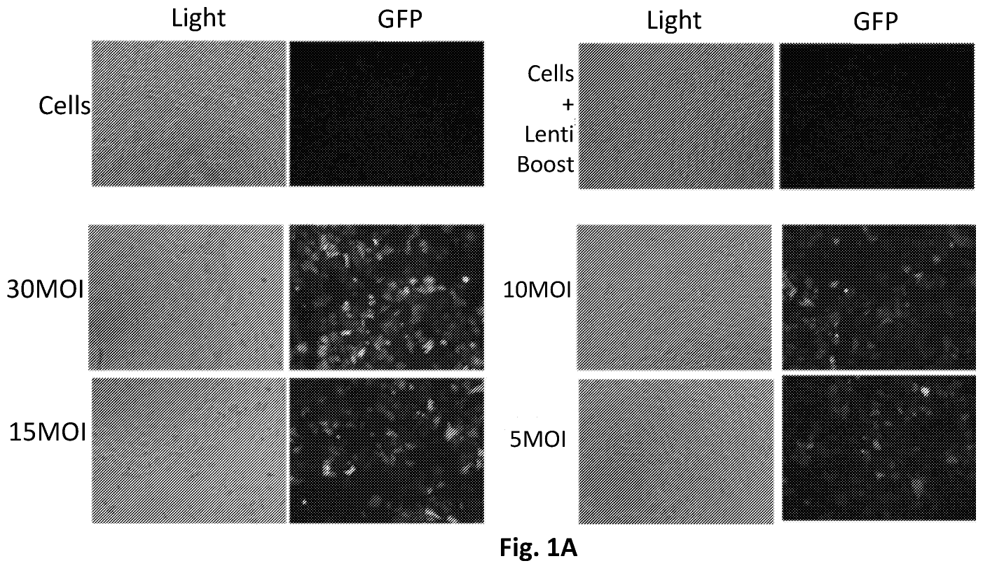

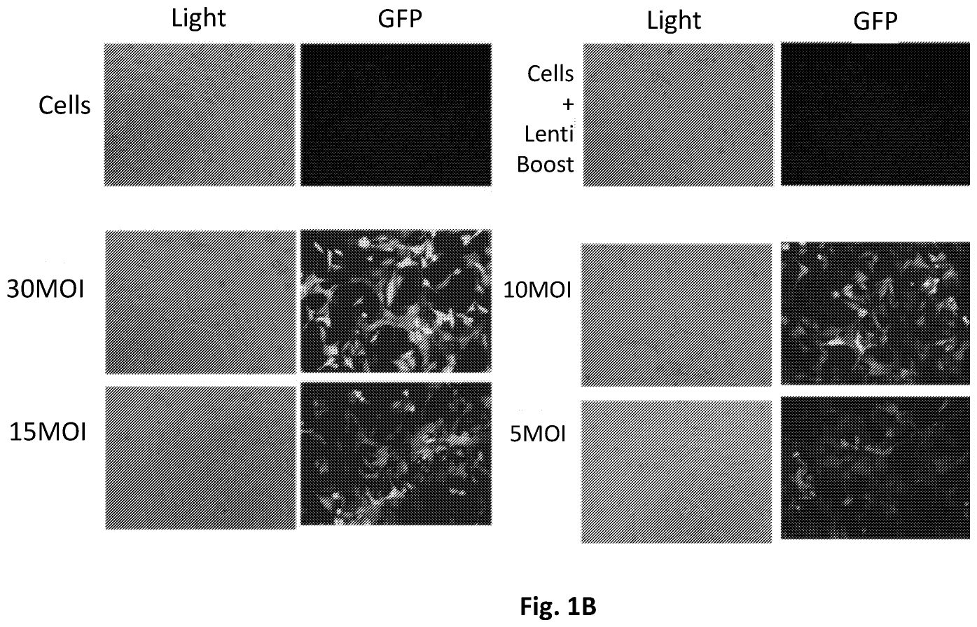

Cell lines “BT549” (breast cancer cells) and “H1975” (non-small-cell lung cancer cells) (5×10³ cells/well) were seeded in 96-well plates. On the next day, LV-scFvCD24 particles, carrying a gene for green fluorescence protein, were added in several multiplicities-of-infections (MOIs) (2, 5, 10, 15, 30) in the presence of lentiboost™ (1:100 buffer A+1:100 buffer B). Cells were then centrifuged for 90 minutes at 800×g at room temperature. After 72 h the efficacy of infection was evaluated qualitatively by florescence microscope. FIG. 1A (Cell line “BT549”) and FIG. 1B (Cell line “H1975”) show representative results.

The results demonstrate that LV-scFvCD24 particles effectively infect both breast cancer cells and NSCL cancer cells in a dose-dependent manner.

### Example 2. In-Vitro Evaluation of Cell Line “H1975” Cell Survival Infected With LV-scFvCD24 Particles

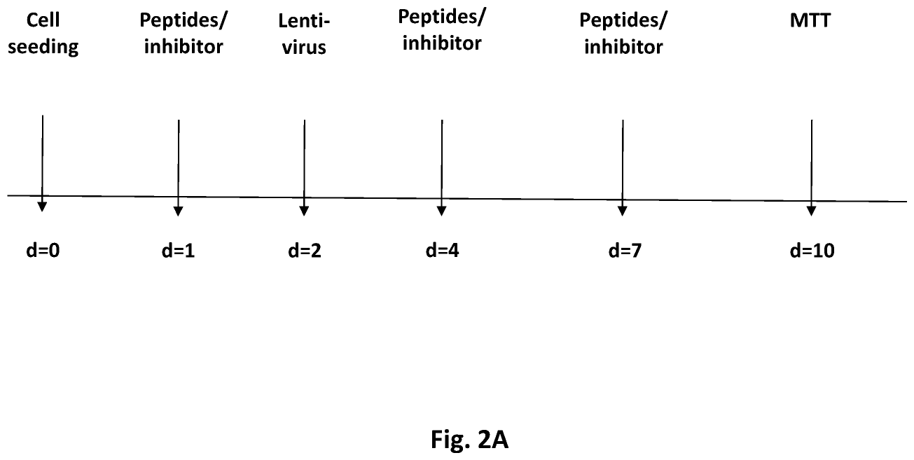

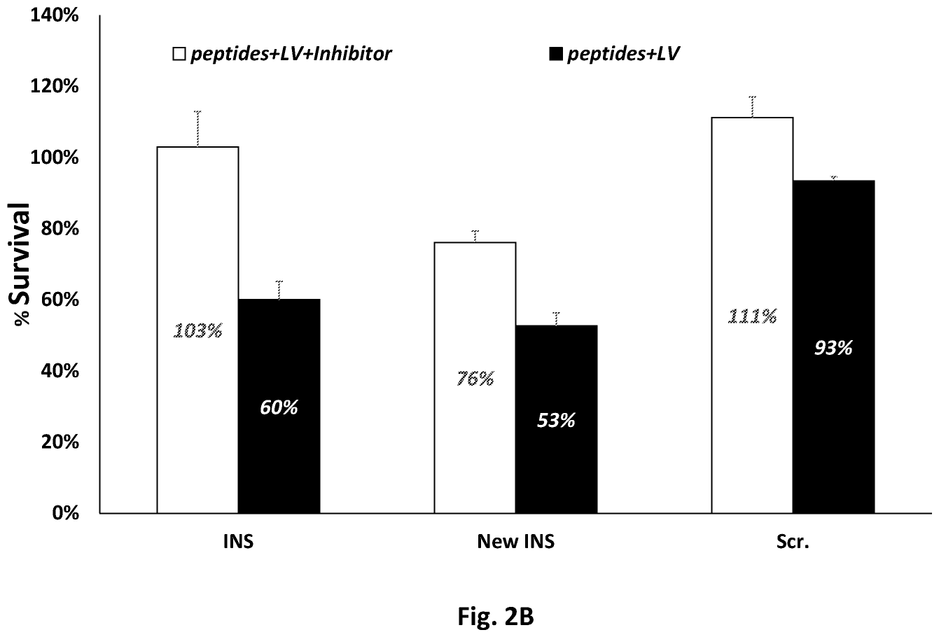

Cell line “H1975” cells were seeded in 96-well plates (2000 cells/well). FIG. 2A shows the timeline of the experiment. INS peptide (in duplicate, “INS” (older batch), “New INS” (fresh batch)) or scrambled peptide (AGTHHWILVTEN, SEQ ID NO:7), with and without Raltegravir inhibitor (“inhibitor”, CAS Number 871038-72-1), was added on days 1, 4 and 7. Particles were added on day 2. At the end of the experiment, cell survival was evaluated by the enzymatic MTT assay (FIG. 2B).

The results demonstrate that combinations of the INS peptide and LV-scFvCD24 particles significantly decreased the survival of lung cancer cells, an effect ameliorated by the antiretroviral drug Raltegravir.

### Example 3. In-Vitro Evaluation of Cell Line “H1975” Infection and Cell Survival With LV-scFvCD24

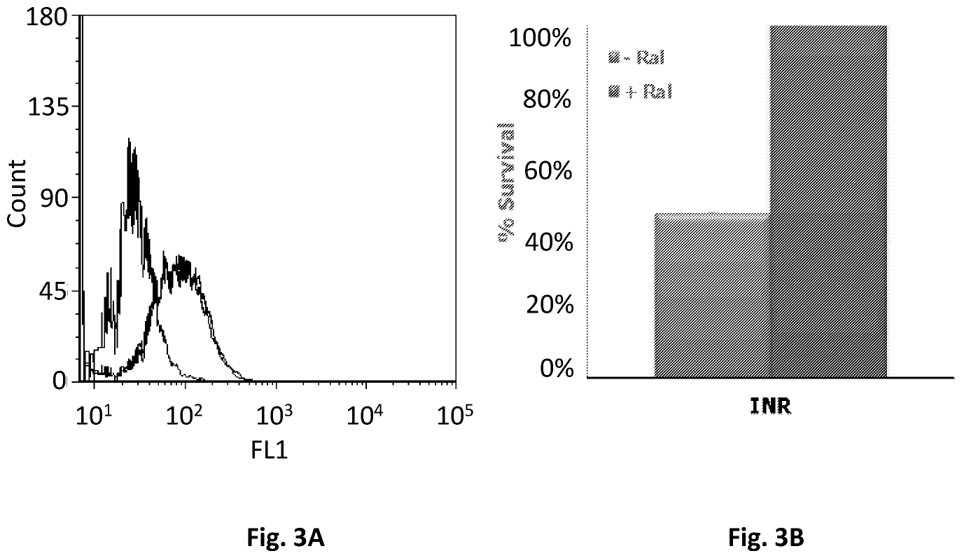

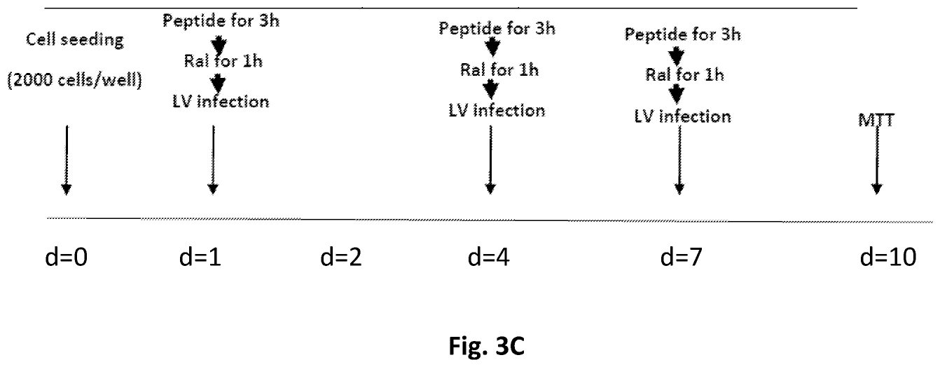

The expression of CD24 in H1975 cells was evaluated by Flow cytometry. Briefly, approximately 1×10⁶ cells were used in each experiment. Fluorescein isothiocyanate (FITC)—labeled humanized anti-human CD24 antibodies were used. Detection of bound antibodies was performed on a Cube6 and results were analyzed with the FCS express program. The lentivirus used was anti-CD24 scFv in an MOI of 30. The LentiBoost™ concentration used was 1:100 buffer A+1:100 buffer B (+spinoculation). The INr2 peptide was used in a concentration of 30 μM. FIG. 3A depicts the fluorescence (count) of the tested cells (Left—negative control, only secondary antibody; Right—duplicates of cells expressing CD24 after first and second-FITC antibodies). FIG. 3B depicts the survival (%) of the tested cells with (right bar) and without (left column) Raltegravir (Ral). FIG. 3C is a schematic illustration of the of the experiment timeline. INr2 peptide, with and without Raltegravir inhibitor (“Ral”), was added on days 1, 4 and 7. Particles were added on days 1, 4 and 7 as well.

The results demonstrate that lung cancer cells indeed express CD24 (the target of LV-scFvCD24 particles), and that a combination of the INR peptide and LV-scFvCD24 particles significantly decreased the survival of these cells, an effect ameliorated by the antiretroviral drug Raltegravir.

### Example 4. In-Vitro Evaluation of Cell Lines “DLD1” and “MCF7” Infection With LV-scFvCD24

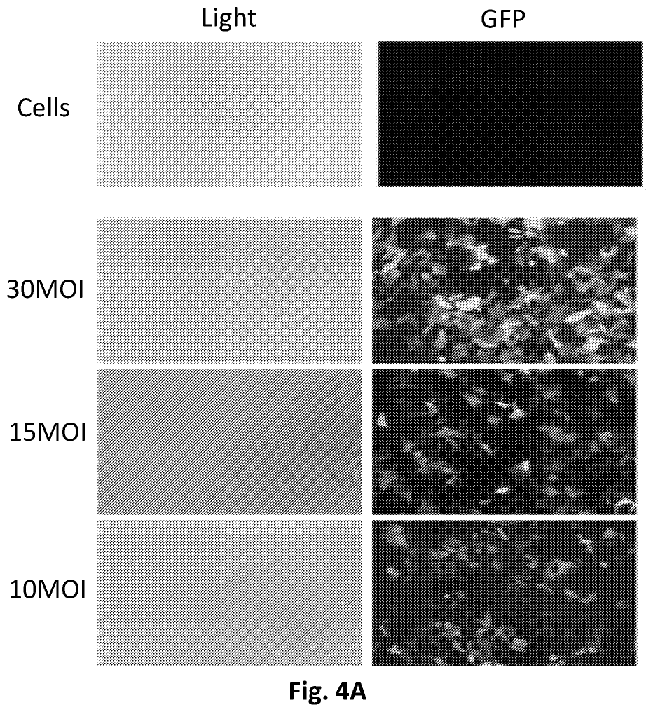

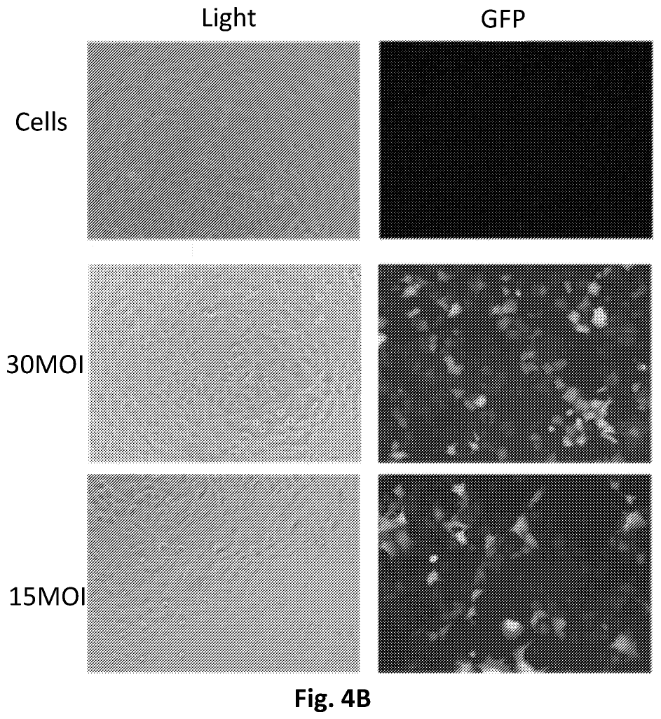

Integrase derived peptides (designated as INS) were synthesized as well as control peptide. Humanized Anti-CD24 antibody fragment (scFv) was engineered and fused to the lentivirus envelope. Cell death was measured qualitatively by using fluorescent microscopy and was quantified by the enzymatic MTT assay. Human colorectal cells (DLD1, colorectal adenocarcinoma, FIG. 4A) and breast cancer cells (MCF7, Human breast adenocarcinoma, FIG. 4B) were used for testing the potency of the lentiviral-based system.

INS was able to stimulate the viral Integrase enzyme in test tubes and in viral infected cells. Massive cell death was induced upon exposure of the infected cells to the INS peptide compared to the control peptide.

The results demonstrate the successful use of IN-derived peptides as integration-promoting agents, together with CD24-targeted lentivirus, and an integrating enzyme, in specifically promoting infection and death of CD24-expressing cancer cells.

### Example 5. In-Vitro Evaluation of the Effect of INS Peptide on the Activity of HIV-1 Integrase

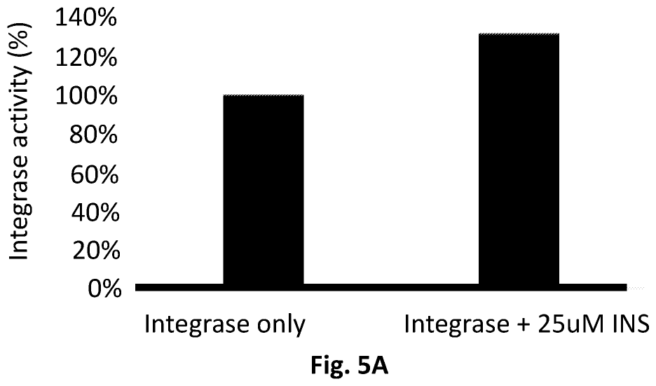

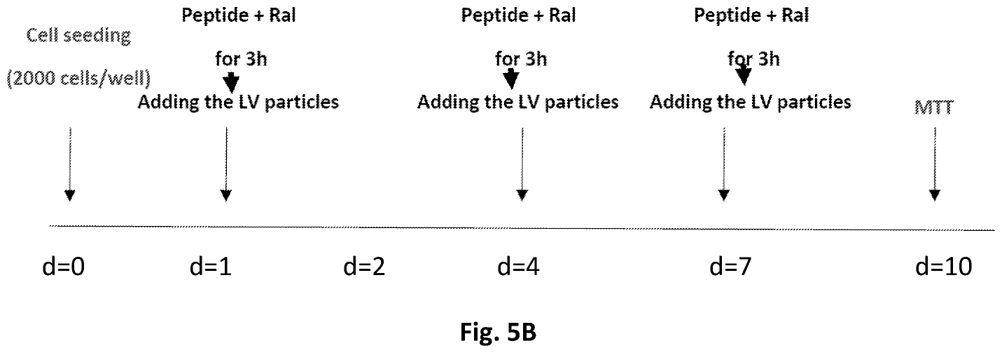

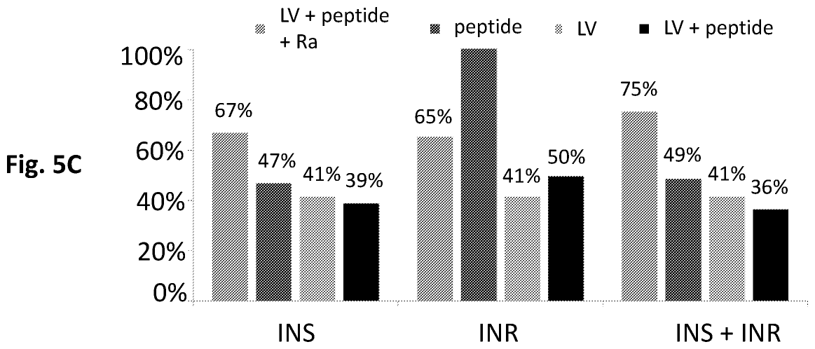

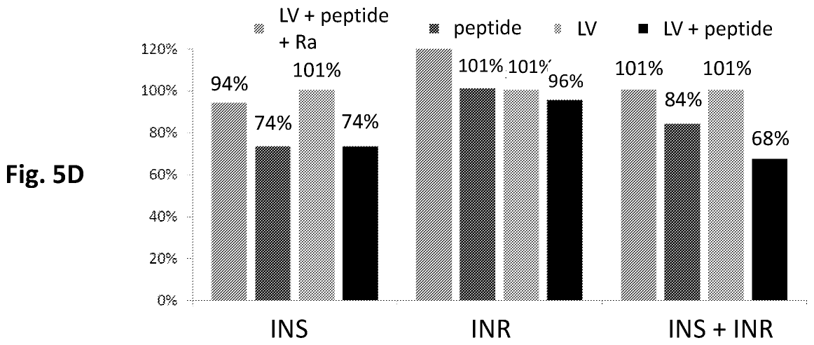

The ability of INS to increase the activity of HIV-1 integrase was tested using the HIV-1 integrase assay kit. FIG. 5A shows relative percent of integrase activity (the peptides were dissolved in DDW). FIG. 5B shows a schematic illustration of the of the experiment timeline.

The effect of peptide activity on survival of 1975 lung cancer cells (FIG. 5C) and BT549 breast cancer cells (FIG. 5D) is demonstrated by the enzymatic MTT assays. Lentivirus—Anti-CD24 scFv fused to the VSV-G virus envelop—30 MOI. LentiBoost Concentration—1:100 buffer A+1:100 buffer B (+spinoculation).

Massive cell death of several cancer cell lines was observed after 10 days of treatment (3 cycles).

The results demonstrate that combinations of the IN-derived peptides and LV-scFvCD24 particles significantly decreased the survival of these cells, an effect ameliorated by the antiretroviral drug Raltegravir.

### Example 6. In-Vitro Examination of Treatment Cancer (Lentiviral Particles & Peptides) on Pancreatic and Lung Cancer Cells

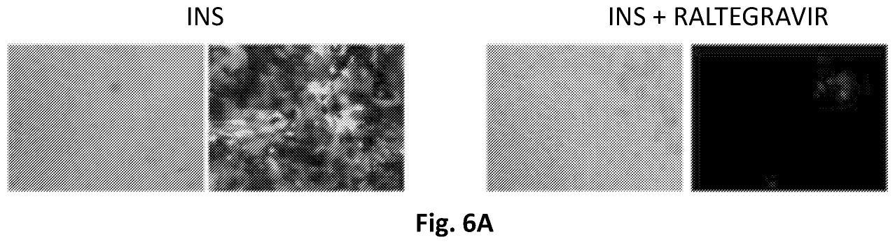

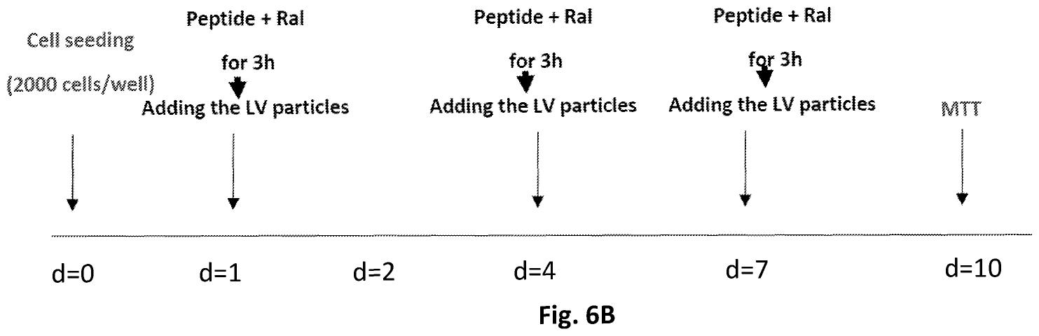

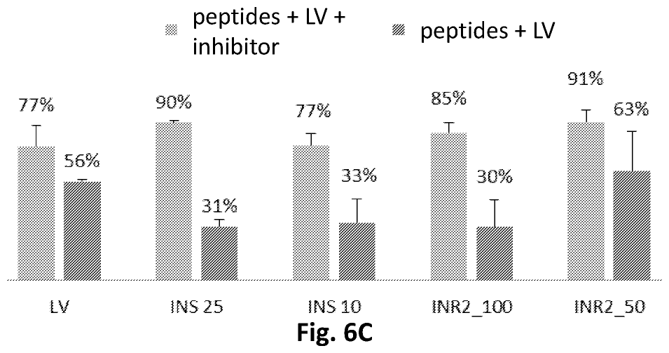

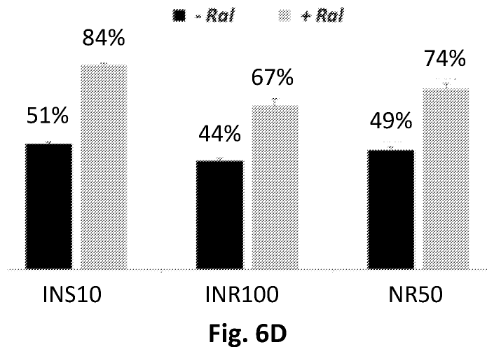

The materials used are:
1. Cell lines—H1975 (non-small-cell lung cancer cells) and Panc-1 (a pancreatic carcinoma of ductal origin),
2. Lentivirus—Anti-CD24 scFv fused to the VSV-G virus envelop, 30 MOI,
3. LentiBoost Concentration—1:100 buffer A+1:100 buffer B (+spinoculation),
4. INR peptide—100 and 50 μM,
5. INS peptide—25 and 10 μM, and
6. Raltegravir—50 μM.
   FIG. 6A demonstrates that Raltegravir inhibits the integrase activity in 1975 cell line. FIG. 6B illustrates the experiment's timeline. The effect of peptides' activity on survival of Panc-1 is demonstrated by the enzymatic WST-1 assay (FIG. 6C), and the effect of peptides' activity on survival of 1975 cells is demonstrated by the enzymatic MTT assay (FIG. 6D).

Massive cell death (60-70%) of cancer cells was observed after 10 days of treatment (3 cycles) in all cancer cell lines tested.

### Example 7. In-Vivo Calibration of the Amount of Lentiviral Particles for Injection in Tumor Mice Model

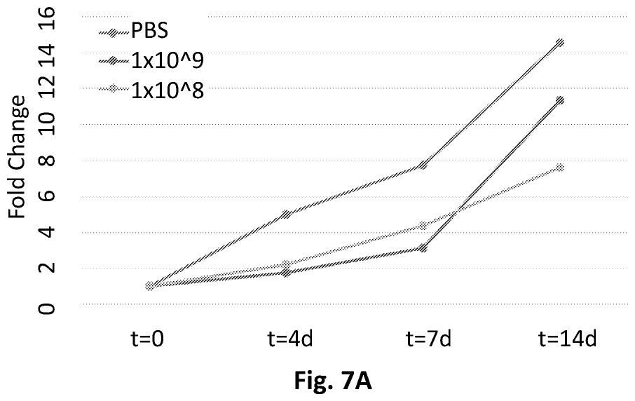

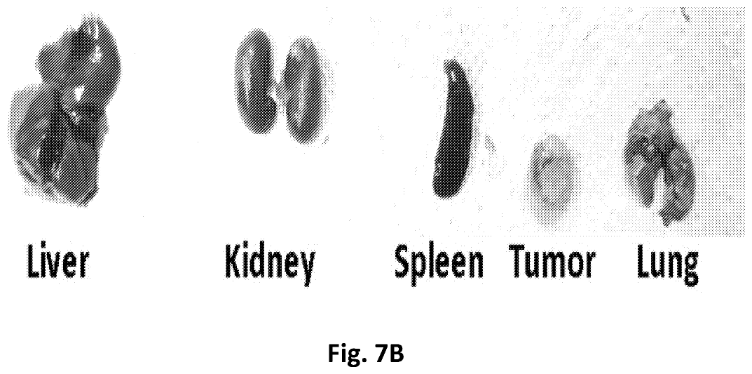

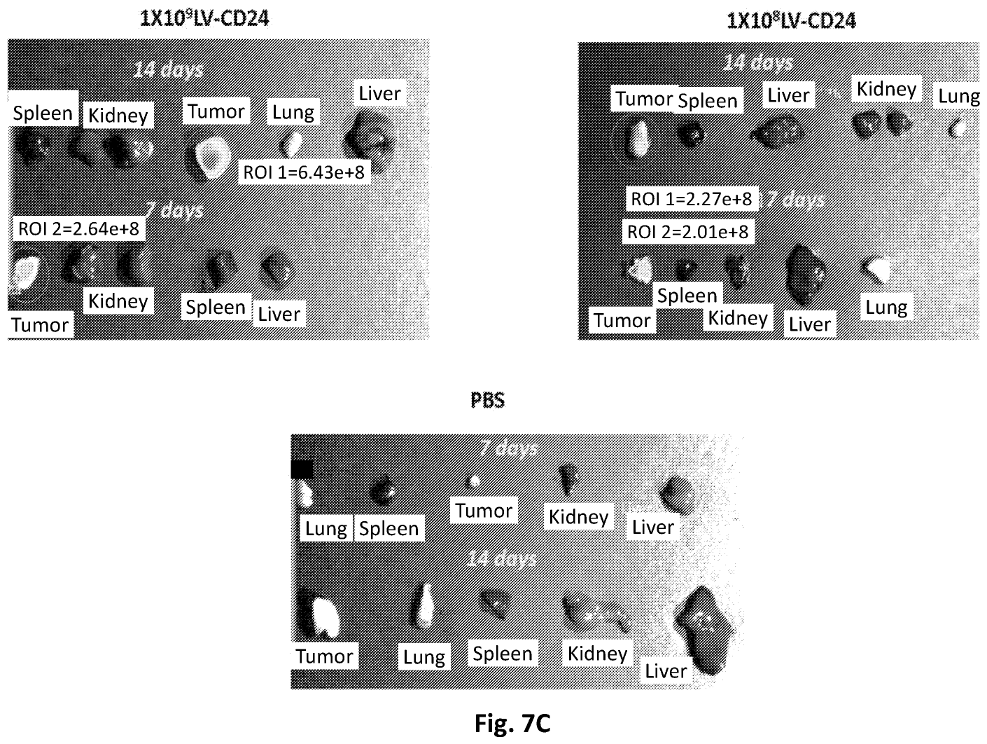

8-weeks old male athymic nude mice (n=6) were housed in sterile cages and handled with aseptic precautions. The mice were fed ad-libitum. For testing the distribution of the lentiviral particles and their possible therapeutic potential, exponentially growing 1975 human lung cancer cells were harvested and resuspended at a final concentration of 5*10⁶ cells per 0.1 ml PBS per injection. The cells were injected subcutaneously at one site on the back of the mice. When tumors were palpable (˜0.3-0.5 cm³), the mice were randomly divided into three groups (PBS, 1*10⁸ IU and 1*10⁹ IU) and the particles were injected. The particles or PBS were administrated via one intraperitoneal injection. Tumor volume, measured with a caliper, was monitored as a function of time from day 0 (FIG. 7A). Tumor volume was calculated as 4/3π·a·b2. At the end of the experiment, the mice were anesthetized and then sacrificed and the tumors were removed, as well as lung, spleen, kidney and liver (FIG. 7B). Lentiviral therapy exhibited a reduction in tumor growth of approximately 50%. GFP expression in tumors and other organs was examined with an IVIS imaging system. As illustrated in FIG. 7C, only tumors, and no other organs, exhibited GFP expression with lentiviral particles in both MOIs tested.

The results demonstrate that the lentiviral particles have therapeutic potential in-vivo, being able to at least significantly slow tumor progression.

### Example 8. In-Vivo Examination of Cancer Treatment in a Tumor Mice Model

Injection of about 5*10⁶ 1975 human lung cancer cells per 0.1 ml PBS per mouse is administrated to 8-weeks old male athymic nude mice (n=56). The cells are injected subcutaneously at one site on the back of the mice. When tumors are palpable (˜0.3-0.5 cm³), the mice are randomly divided into eight groups (PBS, 1*10⁸ LV, INS, 1*10⁸ LV+INS, INr2, 1*10⁸ LV+INr2, INS+INr2, and 1*10⁸ LV+INS+INr2) and the LV particles and/or peptides are injected, twice a week for 3 weeks.

Tumor volume is measured with a caliper. At the end of the experiment, the mice are anesthetized and then sacrificed and the tumors are removed, as well as lung, spleen, kidney and liver. GFP expression is examined by western blot analysis.

### Example 9. Ex-Vivo Examination of Treatment for Leukemia

The materials used are:
1. Cell lines—Primary cells from Acute myeloid leukemia (AML)/chronic lymphocytic leukemia (CLL) patients,
2. Lentivirus—Anti-CD20 scFv fused to the VSV-G virus envelop,
3. LentiBoost Concentration—1:100 buffer A+1:100 buffer B (+spinoculation),
4. INR peptide—50 μM,
5. INS peptide—25 and 10 μM, and
6. Raltegravir—50 μM.

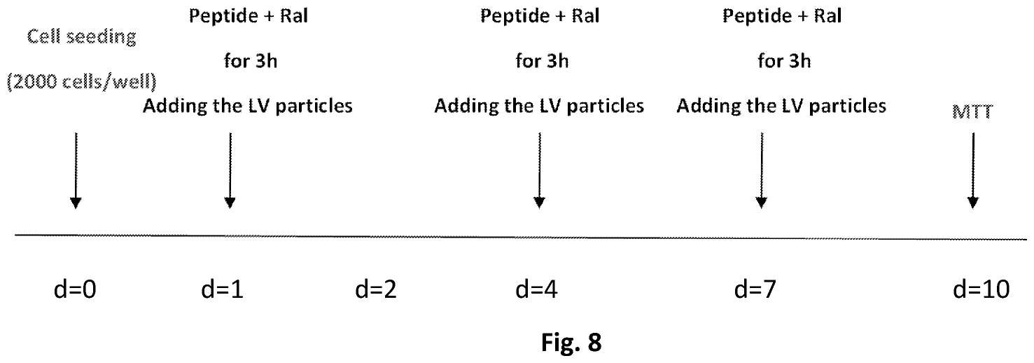

First, lentiviral particles infection of primary cells is tested at different MOI: 0, 2, 5, 10, 15, and 30. GFP expression is used to monitor infection. Then, the effect of treatment of leukemic cells is examined. FIG. 8 is a schematic illustration of the timeline of the experiment.

### Example 10. Attenuation of Tumor Progression in Nude Mice

1-10*10⁶ cancer cells are transplanted subcutaneously in the right hind limb of 6-weeks old nude mice. Tumors are allowed to grow for about 10 days, until they reach an average volume of 50 mm³ as determined by a caliper. On day 0 of treatment, 50-100 μL of saline or 1-10*10⁶ infective HIV-1 derived vector particles targeted to a tumor-specific-antigen presented by the cancer cells are administered systemically or directly into the tumor. A third group receives the same HIV-1 derived vector particles and a combination of INS, INr1 and INr2 peptides. Tumor progression is followed by weekly measurements for at least three months, or until the demise of all mice.

### Example 11. Attenuation of SIV in Immunocompetent Primates

Primates infected by a simian immunodeficiency virus (SIV) are grouped (n=3-6) and administered systemically on day 0 of treatment with 50-100 μL of saline or 1-10*10⁶ infective HIV-1 derived vector particles targeted to SIV gp120. A third group receives the same HIV-1 derived vector particles and a combination of INS, INr1 and INr2 peptides. Viral loads are followed by monthly measurements for at least twenty-four months, or until the demise of all primates.

### Example 12. Weight Loss in Pigs

Pigs are grouped (n=10-15) and administered systemically on day 0 of treatment with 50-100 μL of saline or 1-10*10⁶ infective HIV-1 derived vector particles targeted to an adipocyte marker such as ASC-1, PAT2, and P2RX5 (Ussar et at, “ASC-1, PAT2, and P2RX5 are cell surface markers for white, beige, and brown adipocytes”, Cell Biology, 2014, Vol. 6(247), page 103). A third group receives the same HIV-1 derived vector particles and a combination of INS, INr1 and INr2 peptides. Pig weight is followed by weekly measurements for at least twelve months.

### Example 13. Attenuation of Yeast Infection in Female Cats

1-10*10⁶ yeast cells are administered to the vagina of 6-weeks old female cats. On day 0 of treatment, 50-100 μL of saline or 1-10*10⁶ infective HIV-1 derived vector particles targeted to a yeast-specific-antigen presented by the yeast cells are administered systemically or directly into the vagina. A third group receives the same HIV-1 derived vector particles and a combination of INS, INr1 and INr2 peptides. Infection is followed daily for at least one month.

### Example 14. Attenuation of Bacterial Infection in Immunocompetent Mice

1-10*10⁶ bacteria cells are administered to the eye of 6-weeks old immunocompetent mice. On day 0 of treatment, 50-100 μL of saline or 1-10*10⁶ infective HIV-1 derived vector particles targeted to a bacteria-specific-antigen such as LPS are administered systemically or directly into the eye. A third group receives the same HIV-1 derived vector particles and a combination of INS, INr1 and INr2 peptides. Infection is followed daily for at least one month.

1-56. (canceled)

57. A method for treating a disease or condition in a subject in need of such treatment, by selectively destroying a specific population of cells, the method comprising the step of targeting the cells with a complex comprising:
(i) a linear molecule of double-stranded DNA (dsDNA) comprising long term repeat (LTR) sequences recognized by the integrating enzyme of (ii),
(ii) an integrating enzyme, capable of entering the nuclei of the cells after binding to the LTR sequences of the dsDNA molecule of (i) and creating multiple double strand breaks (DSBs) in the chromosomal DNA of the cells.

58. The method of claim 57, wherein targeting of the cells is achieved by:
i. a targeting moiety capable of binding specifically to a molecule presented by the specific population of cells, included in said complex;
ii. specific expression under a selective promoter, of the dsDNA molecule, the integrating enzyme, or both, in the specific population of cells; or
iii. direct administration of the complex to the specific population of cells.

59. The method of claim 57, further comprising the step of administering to the subject at least one integration-promoting agent.

60. The method of claim 59, wherein the integration-promoting agent is selected from the group consisting of INS peptide (WTAVQMAVFIHNFKRK; SEQ ID NO:1), INr2 peptide (WGSNFTSTTVKA; SEQ ID NO:3), INr1 peptide (WTHLEGKIILVAVHVA; SEQ ID NO:2), LEDGF/p75 protein (UniProt O75475), and any combination thereof.

61. The method of claim 59, wherein the dsDNA molecule encodes the integrating enzyme, the integration-promoting agent, or both.

62. The method of claim 57, wherein the complex comprises a targeting moiety capable of binding specifically to a molecule presented by the specific population of cells.

63. The method of claim 62, wherein the targeting moiety is an antibody or an antigen-binding fragment thereof capable of specific binding to an antigen presented by the cell.

64. The method of claim 63, wherein the antibody or an antigen-binding fragment thereof is an antibody directed to the human cluster of differentiation 24 (CD24).

65. The method of claim 63, wherein the antibody or an antigen-binding fragment thereof is an antibody directed to the human cluster of differentiation 20 (CD20).

66. The method of claim 57, wherein the integrating enzyme is an integrase enzyme selected from the group consisting of HIV-1 integrase, HIV-2 integrase, an active fragment thereof, and an active analog thereof.

67. The method of claim 57, further comprising the step of contacting the cells with:
(i) a transfection-promoting agent capable of increasing the number of the complexes fusing with the cells, or increasing the rate of fusion between the complexes and the cells;
(ii) an apoptosis-promoting agent; or
(iii) an antigenicity-promoting agent selected from the group consisting of a cancer-associated antigen and a pathogen-associated antigen.

68. The method of claim 57, wherein the complex is in a lentivirus particle, a lipid-coated particle or a protein-coated particle.

69. The method of claim 68, wherein the complex is a lentivirus particle comprising a dsDNA, an integrase and a targeting moiety capable of binding human CD24.

70. The method of claim 59, wherein the complex and the integration-promoting agent are administered simultaneously or separately.

71. The method of claim 70, wherein the complex is administered less frequently than the integration-promoting agent.

72. The method of claim 57, wherein the disease or condition is cancer, and the specific population of cells is a population of cancer cells.

73. The method of claim 57, wherein the disease or condition is HIV-infection and the specific population of cells is a population of human immunodeficiency virus (HIV)-infected cells.

74. A pharmaceutical composition, comprising:
(i) a linear molecule of double-stranded DNA (dsDNA) comprising long term repeat (LTR) sequences recognized by the integrating enzyme of (ii), and
(ii) an integrating enzyme, capable of entering the nuclei of cells after binding to the LTR sequences of the dsDNA molecule of (i) and creating multiple double strand breaks (DSBs) in the chromosomal DNA of the cells, or a polynucleotide sequence encoding said integrating enzyme; and
(iii) an integration-promoting agent selected from the group consisting of: INS peptide (WTAVQMAVFIHNFKRK; SEQ ID NO:1), INr1 peptide (WTHLEGKIILVAVHVA; SEQ ID NO:2), INr2 peptide (WGSNFTSTTVKA; SEQ ID NO:3), LEDGF/p75 protein (UniProt O75475), and any combination thereof, or a polynucleotide sequence encoding said integration-promoting agent.

75. A kit, comprising:
(i) a composition comprising a linear molecule of double-stranded DNA (dsDNA) comprising long term repeat (LTR) sequences recognized by the integrating enzyme of (ii), and
(ii) a composition comprising an integrating enzyme, capable of entering the nuclei of cells after binding to the LTR sequences of the dsDNA molecule of (i) and creating multiple double strand breaks (DSBs) in the chromosomal DNA of the cells.

76. The kit of claim 75, further comprising a composition comprising an integration-promoting agent selected from the group consisting of INS peptide (WTAVQMAVFIHNFKRK; SEQ ID NO:1), INr1 peptide (WTHLEGKIILVAVHVA; SEQ ID NO:2), INr2 peptide (WGSNFTSTTVKA; SEQ ID NO:3), LEDGF/p75 protein (UniProt O75475), and any combination thereof.


SEQUENCE LISTING (7 sequences)

SEQ ID NO: 1 - protein, 16 aa, Artificial Sequence
  Synthetic peptide
WTAVQMAVFIHNFKRK

SEQ ID NO: 2 - protein, 16 aa, Artificial Sequence
  Synthetic peptide
WTHLEGKIILVAVHVA

SEQ ID NO: 3 - protein, 12 aa, Artificial Sequence
  Synthetic peptide
WGSNFTSTTVKA

SEQ ID NO: 4 - protein, 15 aa, Artificial Sequence
  Synthetic peptide
TAVQMAVFIHNFKRK

SEQ ID NO: 5 - protein, 15 aa, Artificial Sequence
  Synthetic peptide
THLEGKIILVAVHVA

SEQ ID NO: 6 - protein, 11 aa, Artificial Sequence
  Synthetic peptide
GSNFTSTTVKA

SEQ ID NO: 7 - protein, 12 aa, Artificial Sequence
  Synthetic peptide
AGTHHWILVTEN